(19) United States
(12) Patent Application Publication
(10) Pub. No.: US 2026/0248498 A1
(43) Pub. Date: Aug. 27, 2026

(54) IMPLANTATION TOOL FOR BONE ANCHORS WITH CENTRAL CARTRIDGE MAGAZINE

(71) Applicant: Integrity Orthopaedics, Inc., Maple Plain, MN (US)

(72) Inventors: Nathaniel Van Tran, Lakeville, MN (US); Nathaniel Zenz-Olson, Ham Lake, MN (US); Michael C. Westling, Maple Plain, MN (US); Thomas A. Westling, Orono, MN (US); Ryan D. Majkrzak, Maple Plain, MN (US)

(73) Assignee: Integrity Orthopaedics, Inc., Maple Plain, MN (US)

(21) Appl. No.: 19/543,244

(22) Filed: Feb. 18, 2026

Related U.S. Application Data
(60) Provisional application No. 63/761,728, filed on Feb. 21, 2025.

(51) Int. Cl. A61B 17/04

(52) U.S. Cl. CPC A61B 17/0401; A61B 2017/0409; A61B 2017/0416; A61B 2017/0459; A61B 2017/0464

(57) ABSTRACT

An implantation tool and associated methods for placement of a bone anchor. The implantation tool includes a housing with a handle and a trigger, an anchor tube extending from the housing, an upper slide moveably positioned in the housing, the upper slide including a cartridge opening having a ledge therein, the upper slide moveable between a delivery position and a retracted position, a magazine holding a plurality of cartridges holding anchors therein for implantation, wherein the magazine is centrally located relative to the housing and/or defines positions for holding the cartridge.

### CROSS REFERENCE TO RELATED APPLICATIONS

The present application claims the benefit of and priority to U.S. Provisional Patent Application No. 63/761,728, filed Feb. 21, 2025, titled IMPLANTATION TOOL FOR BONE ANCHORS WITH CENTRAL CARTRIDGE MAGAZINE, the disclosure of which is incorporated herein by reference.

### BACKGROUND

Injuries to connective tissues in the human body are common including various injuries, including tears, at the connection of tendons to bone. For example, the shoulder joint includes the humeral head of the upper arm bone in contact with the indentation of the glenoid working in conjunction with the rotator cuff, which is a combination of muscles and tendons forming a capsule that both stabilizes the joint and causes desired motion. Injury to the connection between tendons of the rotator cuff muscles to the humeral head, usually a tear in a tendon, is common. These tears do not self-heal. It is estimated that in the U.S. over 4 million people annually are referred to a surgeon due to shoulder pain and over 500,000 of these referrals result in shoulder surgery to repair the rotator cuff. Such surgeries may be open or arthroscopic, with trade-offs between the two approaches including longer recovery time and larger scars (open surgery), versus more frequent repair or re-intervention due to sub-optimal success of the tendon repair (arthroscopic surgery).

Enhanced methods for anchoring tendon to bone, including for arthroscopic rotator cuff repair surgery, are desired. In particular, new implantation tools that facilitate the use of a serial array of small anchors are desired.

### OVERVIEW

The present inventors have recognized, among other things, that a problem to be solved is the need for new and/or alternative tools for implantation of a serial array of bone anchors. In some illustrative and non-limiting examples, a high-density array of small anchors, each with an outer diameter in the range of about 3 millimeters (mm) or less may be implanted in correspondingly small bone holes, with relatively close spacing of about 10 mm (or less) hole-center to hole-center. The anchors of the high-density array are each linked to a working suture. Further, each anchor is associated with a suture lock that secures the working suture to the anchor. The result is a series of independent anchor-to-anchor stitches. With such a system, an anchor implantation tool is desired that will allow the physician to form a bone hole, maintain registration of the tool with the bone hole while passing an anchor into the bone hole, and secure the anchor in the bone hole and activate the suture lock, all while managing the working suture, suture locks, and other anchors.

An implantation tool for delivering bone anchor to a target location is disclosed. The implantation tool is used with a magazine that holds a plurality of cartridges, with each cartridge holding an anchor for delivery with the implantation tool. The magazine is held on the implantation tool in a central location, making for a convenient and relatively easy-to-use profile for the implantation tool and magazine.

In an illustrative example, the magazine includes a front stop against which cartridges in the magazine are pressed by a spring contained in the magazine. The frontmost or “distal-most” cartridge in the magazine can be in either a raised position or a depressed position, the depressed position being used for delivery of the anchor in the cartridge to a target site. The magazine includes a front stop that engages the distalmost cartridge to limit movement from the raised position to the depressed position until such movement is desired by the physician using the implantation tool.

The magazine may include a magazine opening through which the cartridge can be depressed to pass from the raised position to the depressed or delivery position. The magazine opening, in turn, is aligned with a cartridge opening in the housing. The cartridge may further include a side protrusion. The side protrusion may engage with a side opening in the cartridge opening so that the depressed or delivery position of the cartridge is maintained until release of the cartridge is desired.

A first illustrative and non-limiting example takes the form of an implantation tool for placement of a bone anchor array comprising: a housing with a handle and a trigger; an anchor tube having an anchor tube proximal end and an anchor tube distal end, the anchor tube proximal end attached to the housing; an upper slide moveably positioned in the housing, the upper slide including a cartridge opening having a ledge therein, the upper slide moveable between a delivery position and a retracted position; a magazine holding a plurality of cartridges including at least a first cartridge and a second cartridge, the first cartridge being distal of the second cartridge in the magazine, each cartridge having a shoulder at a lower portion thereof and an anchor space open at proximal and distal ends, the anchor space containing an anchor of the bone anchor array, the upper slide configured to receive the magazine such that the distalmost cartridge is positioned over the cartridge opening, wherein two positions are defined for the distalmost cartridge: a first position in which the shoulder is positioned above the ledge; and a second position in which the distalmost cartridge is depressed into the cartridge opening with the shoulder abutting the ledge.

Additionally or alternatively, the magazine includes a front stop, a plunger and a spring, the spring urging the plunger distally to press the cartridges toward the front stop.

Additionally or alternatively, the front stop comprises a cartridge retainer and each cartridge includes a cartridge indent, the cartridge retainer positioned to engage the cartridge indent of a distalmost cartridge in the magazine to hold the distalmost cartridge in the magazine in the first position until the distalmost cartridge in the magazine is manually depressed into the second position.

Additionally or alternatively, each cartridge has a distal side with a cartridge indent, and a proximal side with a cartridge projection, positioned such that the cartridge projection of the first cartridge is engaged with the cartridge indent of the second cartridge to hold the first cartridge in the first position until manually depressed into the second position.

Additionally or alternatively, each cartridge has a distal side with a cartridge projection, and a proximal side with a cartridge indent, positioned such that the cartridge indent of the first cartridge is engaged with the cartridge projection of the second cartridge to hold the first cartridge in the first position until manually depressed into the second position.

Additionally or alternatively, each cartridge in the magazine is removeable from the magazine when in the second position, and the spring and plunger are adapted to apply pressure to hold a distalmost remaining cartridge against the front stop as each successive cartridge is removed from the magazine.

Additionally or alternatively, the anchor space is generally cylindrical having a top slot.

Additionally or alternatively, when the first cartridge is in the second position, the first cartridge blocks the distal end of the anchor space of the second cartridge.

Additionally or alternatively, the housing has a distal end at which the anchor tube is attached, and a proximal end opposed to the distal end, and width, and the magazine has a width, and the width of the magazine is less than the width of the housing.

Additionally or alternatively, the magazine is centrally located within the width of the housing.

Another illustrative and non-limiting example takes the form of a method of placing an anchor in a bone using an implantation tool, the method comprising: using an implantation tool having a housing with a proximal end and a distal end, with an anchor tube extending from the distal end of the housing to an anchor tube distal tip, an upper slide moveable relative to the housing and adapted to receive a magazine containing cartridges, the upper slide having a cartridge opening and a first lumen extending proximally from the cartridge opening and a second lumen extending distally from the cartridge opening, and a bone punch having a bone punch distal tip, a shaft, a bone punch latch, and a bone punch handle, placing a magazine in the upper slide to position a cartridge held by the magazine over the cartridge opening; positioning the distal tip of the anchor tube at a desired location for placing the anchor; advancing the bone punch into the anchor tube until the bone punch distal tip exits the bone punch and forms a bone hole; retracting the bone punch from the bone hole a first time, through the anchor tube and into the housing until the bone punch distal tip is in a lumen of the upper slide proximal of the cartridge opening; depressing the cartridge into the cartridge opening; advancing the bone punch through the cartridge to push the anchor from the cartridge through the anchor tube and into the bone hole; and retracting the bone punch from the bone hole a second time.

Additionally or alternatively, the cartridge holds the anchor in an anchor space having a proximal open end and a distal open end, and the step of depressing the cartridge into the cartridge opening positions the proximal open end in alignment with the first lumen of the upper slide, and the open distal end in alignment with the second lumen of the upper slide.

Additionally or alternatively, the upper slide includes an inner tube attached thereto, the inner tube extending distally from the upper slide into the anchor tube and having an inner tube lumen aligned with the second lumen of the upper slide, such that the step of advancing the bone punch through the cartridge to push the anchor from the cartridge through the anchor tube and into the bone hole includes pushing the anchor through the inner tube lumen.

Additionally or alternatively, the upper slide is moveable relative to the housing, such the inner tube is moveable relative to the anchor tube, wherein: advancing the bone punch into the anchor tube until the bone punch distal tip exits the bone punch and forms a bone hole includes advancing the upper slide relative to the housing and advancing the inner tube relative to the anchor tube such that the inner tube extends into the bone hole; retracting the bone punch from the bone hole a first time is performed without removing the inner tube from the bone hole; and retracting the bone punch from the bone hole a second time includes removing the inner tube from the bone hole.

Additionally or alternatively, the anchor has at least one bore through which a working suture passes and at least one bore through which a suture lock extends, the suture lock having a loop extending around the working suture and a free end secured to the cartridge, the cartridge containing a spool on which the free end is wound.

Another illustrative and non-limiting example takes the form of a combination cartridge, bone anchor for implantation in a bone, and suture lock, the suture lock having a free end and a locking loop, the cartridge configured for holding the bone anchor for implantation in a bone, the cartridge comprising a body having a distal side, a proximal side, and an anchor space extending from an open end at the distal side to an open end at the proximal side, the anchor space having a slot extending upward therefrom toward an upper end of the body, the cartridge holding therein a spool for receiving the free end of a suture lock, the bone anchor having a bore therethrough having a first end and a second end, wherein the suture lock is positioned in the bore with the locking loop extending out of the first end of the bore and the free end extending up to the gap and then to the spool out of the second end of the bore.

Additionally or alternatively, the cartridge has a cartridge gap along one side of the slot, and a protrusion extending laterally from the cartridge adjacent the cartridge gap, the protrusion for securing a position of the cartridge relative to an implantation tool for use therewith.

Additionally or alternatively, the cartridge is configured to be held in a magazine, the magazine having an upper slot, the cartridge having a neck sized to fit in the upper slot, the spool being positioned above the neck.

Additionally or alternatively, the cartridge includes an indent configured to interact with either another cartridge or a front stop of a magazine to hold the cartridge in a first position therein until manually depressed to a second position.

Additionally or alternatively, the cartridge has a shoulder at a lower portion thereof, the shoulder configured to limit downward movement of the cartridge into a housing of an implantation tool configured for use with the cartridge.

This overview is intended to introduce the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an example implantation tool;

FIG. 2 is a perspective view of an example implantation tool without a handle;

FIG. 3 is a perspective view of an example implantation tool with several parts removed and a retracted bone punch;

FIGS. 4A-4B show a detail view and a section view the tool handle;

FIGS. 5A-5D show section views of a portion of an example implantation tool in first and second configurations;

FIGS. 6A-6B show a perspective view and a section view of an upper slide;

FIGS. 7A-7B show upper and lower perspective views of a lower slide;

FIG. 8 is a top-down section view of an example implantation tool;

FIGS. 9 and 10 are front and rear partial exploded views of a magazine, upper slide and lower slide;

FIGS. 11A-11C show an illustrative process of using the trigger;

FIGS. 12A-12E show perspective and cut-away views of the magazine with cartridges;

FIG. 12F is a rear elevation view of the implantation tool;

FIGS. 13-16 show a cartridge with anchor, working suture and locking suture;

FIGS. 17-22 illustrate a step by step process of using the implant tool;

FIG. 23 show an illustrative anchor;

FIG. 24 is a section view of the anchor with working suture and locking suture;

FIG. 25 shows a serial array of implanted anchors; and

FIGS. 26A-26H illustrate the implant tool being used on a tendon and bone.

### DETAILED DESCRIPTION

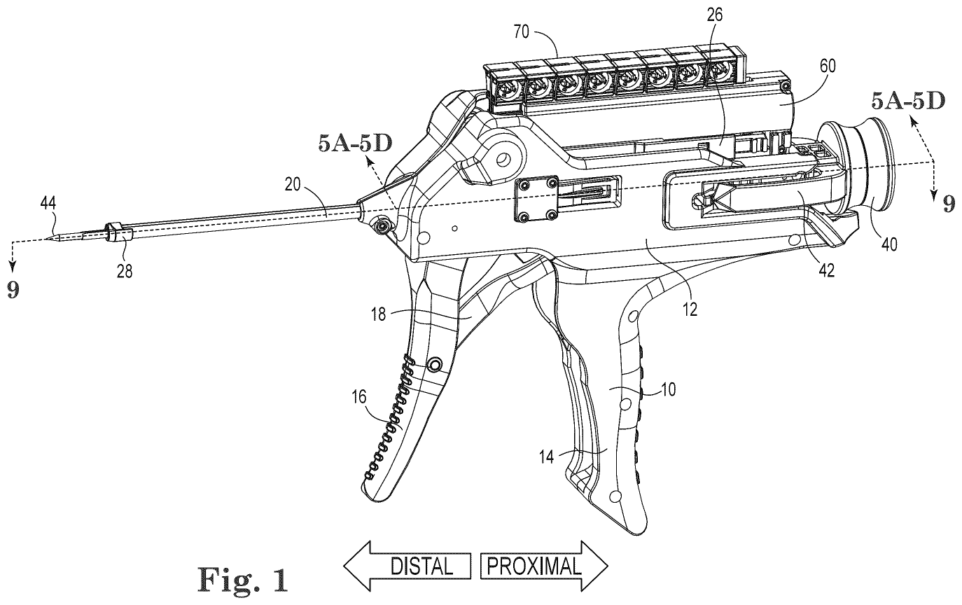

FIG. 1 is a perspective view of an example implantation tool. The implant tool 10 includes a housing 12 which is formed with a handle 14. For example, the housing 12 and handle 14 may comprise first and second halves which are held together by a plurality of screws, rivets or other attachment devices.

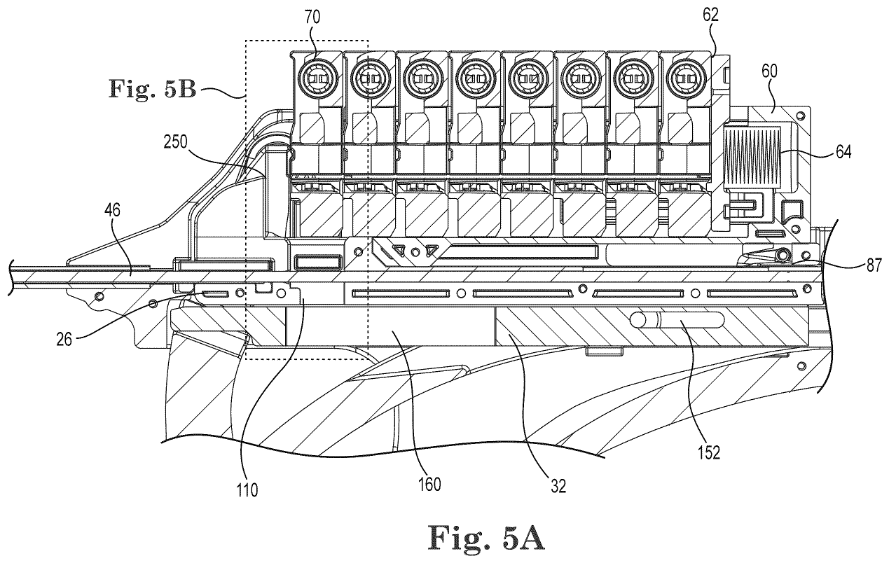

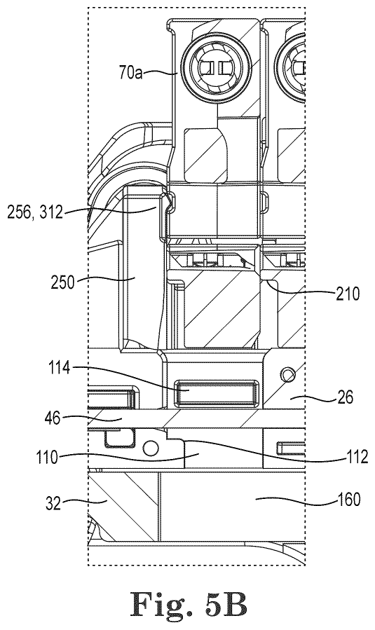

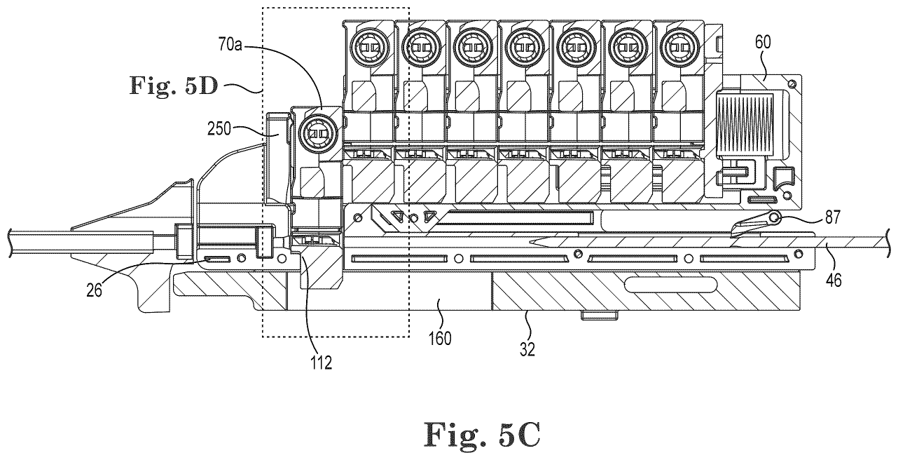

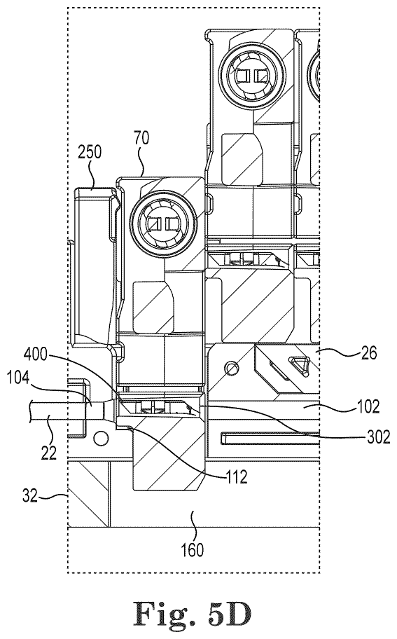

The housing 12 is pivotably coupled to a trigger 16. The trigger 16 connects to a lower slide 32 by a trigger link 18. When the trigger 16 is pulled, in initial movement the trigger acts on the lower slide to push the lower slide in the proximal direction (as marked with the arrow) and, as the trigger action continues, the trigger link 18 pushes the lower slide 32 still further in the proximal direction relative to the handle 14, as illustrated in detail in FIGS. 1A-11C. Depending on the position of a cartridge 70, an upper slide 26 may also move along with the lower slide 32, as detailed further with respect to FIGS. 5A-5D.

As used herein, a “slide” refers to the structural piece shown in the drawings. A slide can be formed of any suitable material, including biocompatible and/or non-toxic plastics. The upper and lower slide are each, as indicated by their names, adapted for movement or “sliding” motion within the housing relative to one or more of the housing, the other slide, or other componentry.

The housing 12 has an anchor tube 20 attached thereto, extending distally from the housing 12. The attachment of the anchor tube 20 to the housing 12 may be achieved mechanically, by providing an indent or opening in the anchor tube which mates with a projection inside a lumen at the housing, for example, though other attachment (welding, adhesive, insert molding, etc.) may be used instead. The anchor tube 20 is a tube of any suitably durable and stiff material, such as stainless steel, for example and without limitation, and may be cylindrical or other shape, such as elliptical, as desired. The anchor tube 20 has an anchor tube tip 28, which is used to help control sutures used during anchor implantation.

A bone punch 40 is provided. The bone punch 40 includes a bone punch latch 42, which selectively engages the housing 12. The bone punch 40 is shown in FIG. 1 as fully inserted, so that at the distal end of the assembly, a bone punch tip 44 extends beyond the distal end of the anchor tube 20.

The tool 10 is shown carrying a removeable magazine 60 which carries a plurality of cartridges 70 (later Figures may show the distalmost cartridge as 70a and others as 70b). Each cartridge 70 carries an anchor and a locking suture associated with the anchor. Some examples may include a single working suture extending through each anchor in the set of cartridges, passing from one cartridge to the next within the magazine 60.

Throughout the remaining discussion, the directions proximal and distal will be used consistent with how each is shown in FIG. 1.

Any suitable material may be used for the componentry shown herein, recognizing that the device and system are to be used for placing bone anchors in a patient, and so biocompatible and sterilizable materials suitable for a device that contacts blood and/or other bodily fluids/tissue should be used. The anchor tube and/or inner tube may be made of stiff materials such as stainless steel or other metal, as desired, though plastic may be used as well. Typically the remaining components will be made of stiff and/or hard biocompatible plastics.

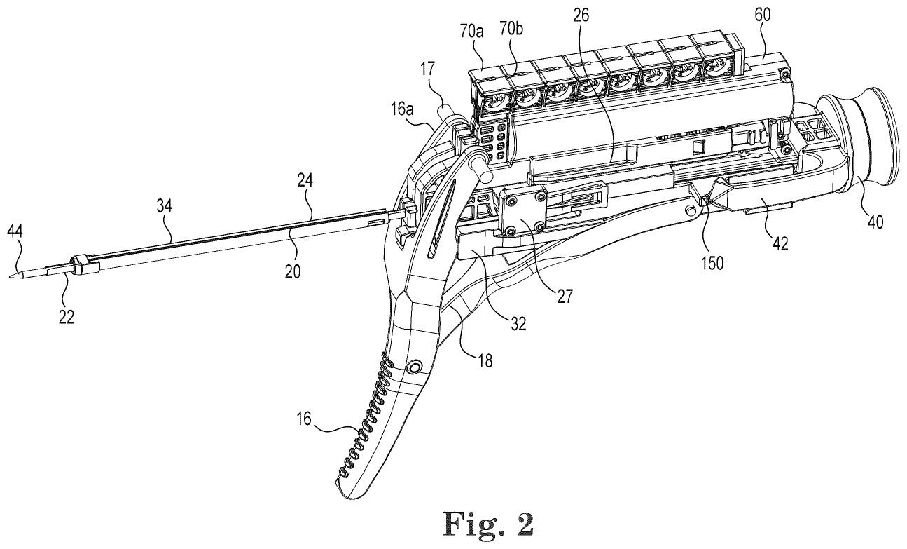

FIG. 2 is a perspective view of an example implantation tool without a handle. The handle and housing are omitted to allow other components to be more readily observed. The trigger 16 can be seen to include a trigger fork 16a that is coupled to a trigger pivot 17, which would in turn be secured inside the housing (not shown).

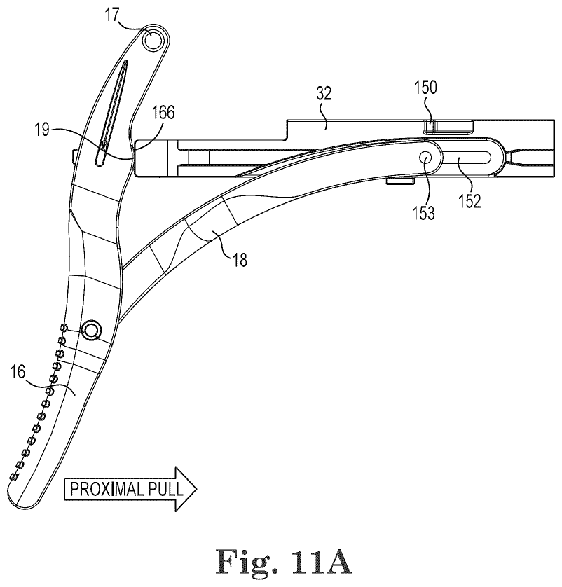

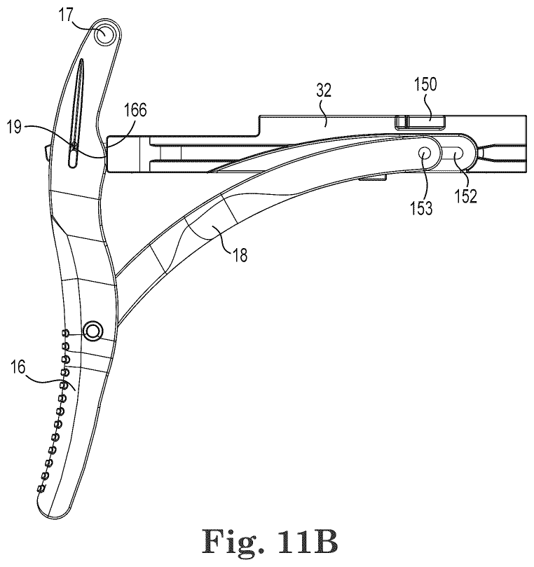

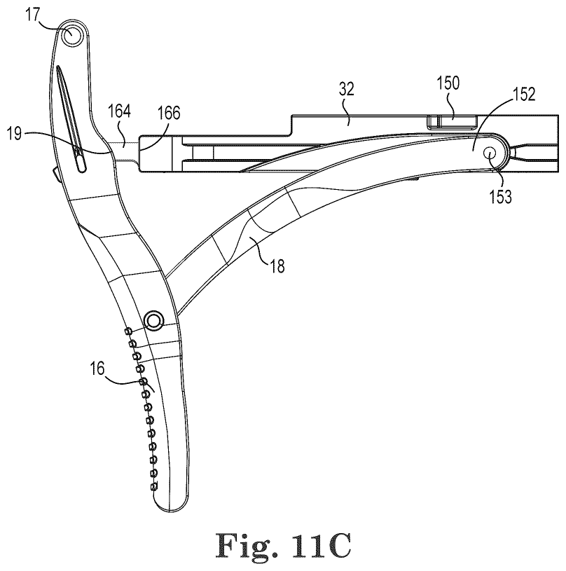

As shown in FIG. 2, the anchor tube 20 includes an anchor tube slot 24. An inner tube 22 extends within the anchor tube 20, and is fixed at its proximal end to an upper slide 26. The upper slide 26 is positioned over and adjustably mates with a lower slide 32. As indicated by their names, the upper slide 26 and lower slide 32 are both configured to slide within the housing as well as relative to one another. The trigger link 18 is secured to the lower slide 32 in a limited sliding manner as explained further in relation to FIGS. 11A-11C.

The inner tube 22 has an inner tube slot 34 that extends the length of the inner tube 22, parallel to and open to the anchor tube slot 24. The alignment of these slots allows a suture to pass therethrough as an implantable anchor is passed down the inside of the inner tube 22. The inner tube is secured at its proximal end to the upper slide 26.

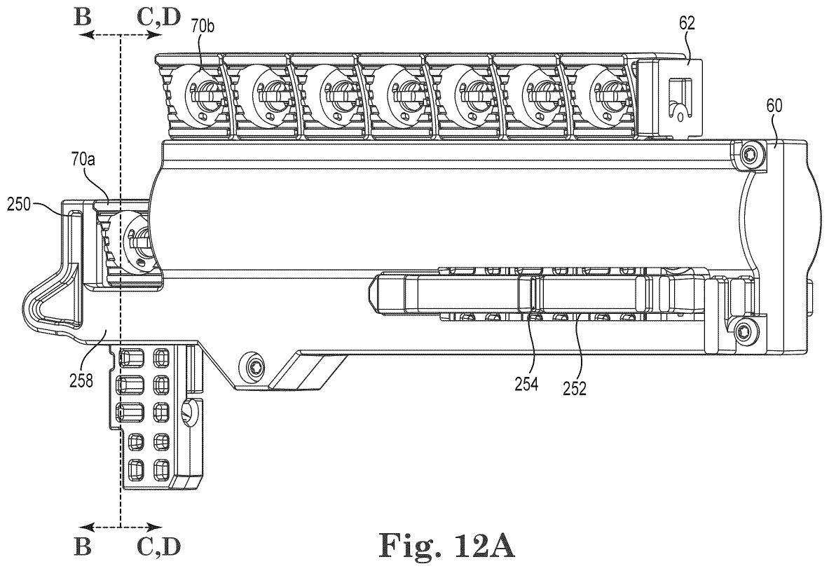

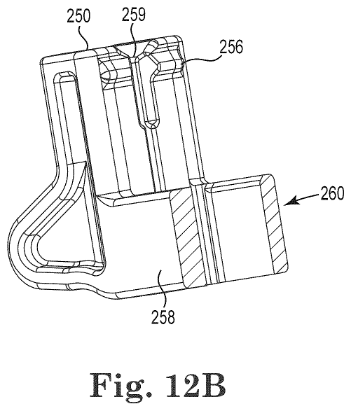

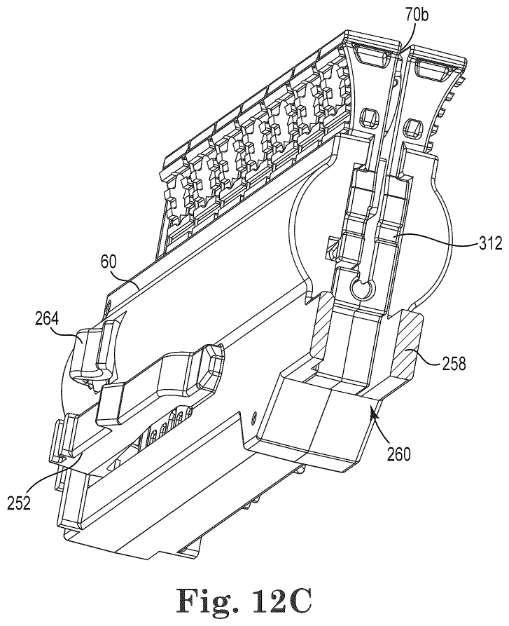

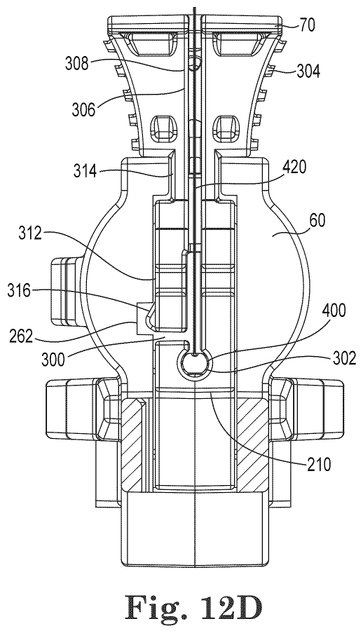

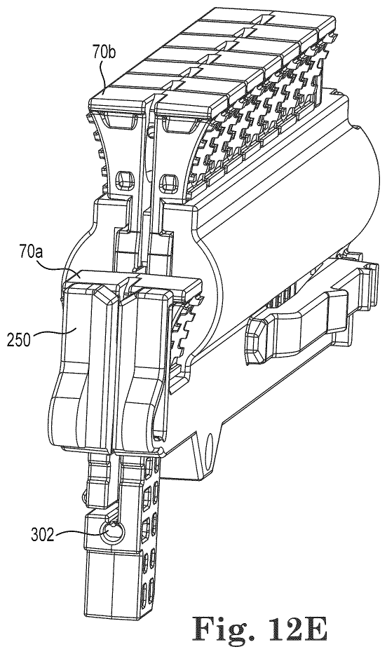

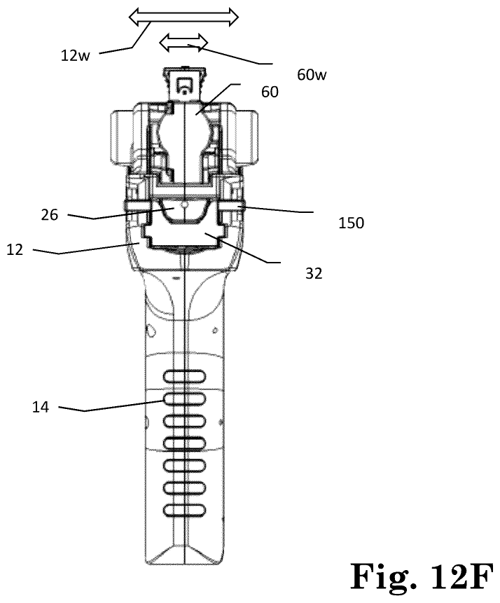

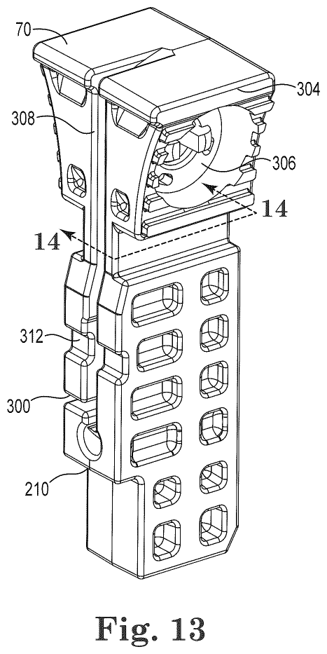

The upper slide 26 has a magazine 60 releasably secured thereto, with the magazine 60 in turn carrying at least one cartridge 70a, 70b (collectively, cartridges 70), each of which contains an anchor to be implanted as well as features to manage a suture and suture lock, as further detailed with reference to FIGS. 13-16. The magazine 60 slides into the position shown relative to the upper slide, and, optionally, clicks into place with holding features along the sides thereof, discussed with reference to FIGS. 12A-12C. When the upper slide 26 has the magazine 60 secured therein, the inner tube 22, upper slide 26, and magazine 60 will all slide together as a single unit, providing a fixed relationship so that an anchor in a cartridge 70a, depressed to a delivery position, will reliably pass or transfer the anchor from the cartridge into the inner tube 22 as the bone punch is advanced in a distal direction.

The bone punch 40 is held to the housing (not shown) by the bone punch latch 42. The lower slide 32 includes a bone punch release tab 150 which is used to spread the bone punch latch 42 to release from the housing when the lower slide 32 is retracted in a proximal direction using the trigger 16, as illustrated in FIGS. 11A-11C.

An upper slide latch 27 is shown as well. The upper slide latch 27 is secured to the housing (not shown) and applies friction against the sliding movement of the upper slide, reducing inadvertent movement. The friction against sliding movement can range from enough to prevent sliding movement due to gravity to a dimple or recess that provides releasable latching of the upper slide relative to the housing.

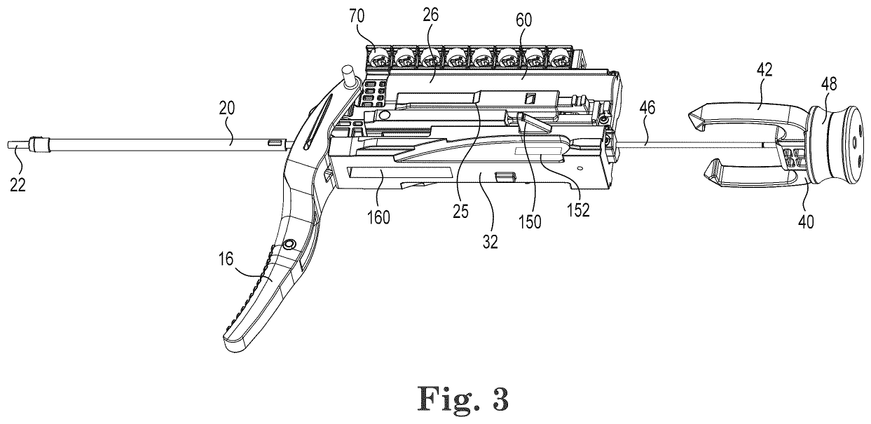

FIG. 3 is a perspective view of an example implantation tool with several parts removed. The bone punch 40 is retracted in a proximal direction relative to the rest of the components. The bone punch 40 includes a bone punch handle 48 having a generally flat proximal surface to allow a physician to tap or pound on the bone punch while creating a bone hole using the physician's hand or a hand tool, such as a mallet. The bone punch latch 42, comprising opposing flexible arms, extends distally from the bone punch handle 48, and the bone punch shaft 46 extends toward the distal end and distal tip of the bone punch (not shown).

The view in FIG. 3 is from below so that the trigger link slot 152 on the lower slide can be more easily seen. The lower slide 32 also includes a lower slide slot 160. The distal end of the lower slide slot 160 aligns with a distal side of the cartridge 70, such that the distal-most cartridge, when depressed, will extend into the lower slide slot 160.

Toward the distal end of the tool, it can be observed that with the upper slide 26 in a distal position, the distal end of the inner tube 22 extends beyond the distal end of the anchor tube 20. The slot of each of the inner tube 22 and anchor tube 20 extends along a top side thereof, and is not visible in the view from below.

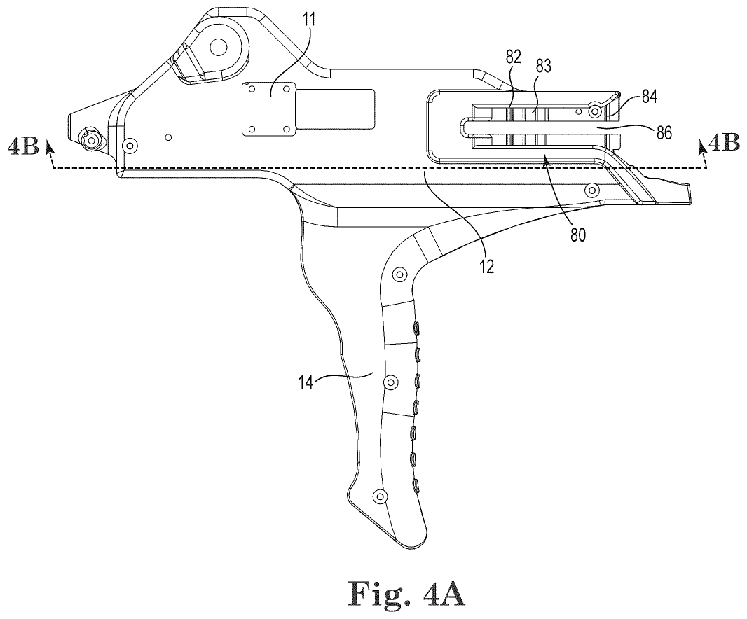

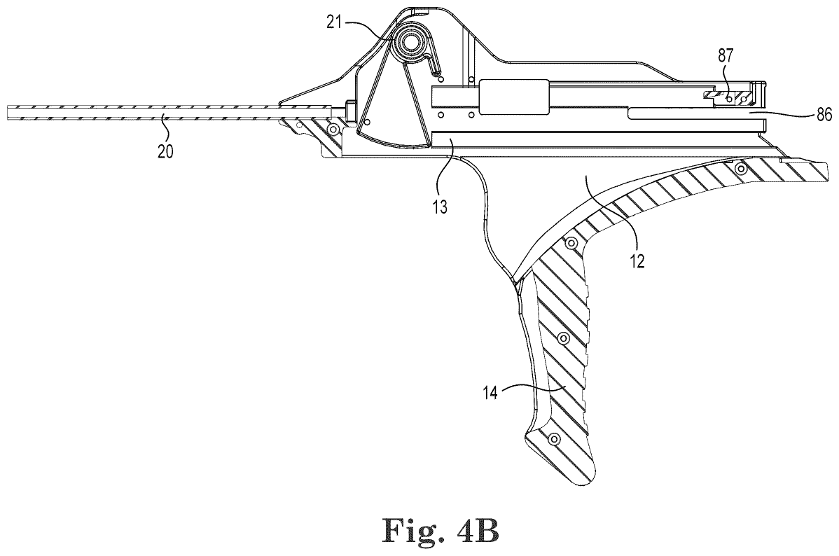

FIGS. 4A-4B show a detail view and a section view of the housing 12 and handle 14. The handle includes an upper slide latch receiver 11, which is configured to receive and secure the upper slide latch 27 (FIG. 2). The handle 14 also has a latch receiver shown at 80. The latch receiver 80 includes a distal detent 82, a middle detent 83, and a proximal detent 84. The bone punch latch is designed to engage with each of these detents 82, 83, 84 as it is advanced in a distal direction. In use, when the bone punch is advanced distally, the engagement with the proximal detent 84 provides the physician with tactile and/or audible feedback that the bone punch distal end is nearing the distal end of the anchor tube. Such feedback may remind the physician to confirm that the sutures used with the anchor are appropriately tightened and arranged for further advancement thereof.

When the bone punch is nearly fully inserted, the bone punch latch engages the middle detent 83. This position will have the distal tip of the bone punch extending through and beyond the distal end of the anchor tube. The inner tube may also extend beyond the distal end of the anchor tube when the middle detent 83 is engaged by the bone punch latch. Such a position is useful for probing and ensuring desired positioning of the bone punch prior to making a new bone hole. For example, in a smaller space, such as a patient with smaller anatomy, this “middle” position with the sharp bone punch tip at less than full depth allows the physician user to move to tool more freely.

When the physician has selected a location for the bone hole, the physician will press the tool against tissue at the desired position, which may be within or outside of the outer margin of the tendon (or other tissue) that is to be attached to the bone. The physician will then tap against the bone punch handle to advance the tip of the bone punch through the tendon or other tissue (if working within the tissue margin) and then into bone. As this tapping continues, the distal end of the anchor tube will press against the tissue, and the continued application of force will push the bone punch distally until the bone punch latch engages with the distal detent 82. In this position, the inner tube extends beyond the distal end of the anchor tube, preferably into the formed bone hole, so that as the implantation procedure continues, the physician can maintain registration of the lumen within the inner tube with the bone hole. In an alternative method, the user can fully advance the bone punch and inner tube prior to applying force to form the new bone hole.

The open slot 86 can be seen as well. The bone punch release tab of the lower slide extends out through this open slot 86, with a resting position at the distal end of the open slot 86. When the trigger is actuated, the action of the trigger (FIGS. 11A-11C) pushes the lower slide in a proximal direction, causing the bone punch release tab to move proximally within the open slot from its resting position to press against the bone punch latch, releasing the bone punch latch from each detent as the trigger continues to be actuated and the lower slide moves in a proximal direction.

FIG. 4B is a cut-away view of the housing 12 and handle 14. In some examples, a two piece housing and handle is used, with left and right halves; here, the inner details of one half are generally shown. A lower slide slot 13 provides a channel within which the lower slide moves. A trigger spring 21 is used to bias the trigger in the distal direction, thereby defining the resting position of the lower slide. A punch stop 87 is provided above the open slot, and includes a spring-loaded stop that will engage with an indentation on the bone punch shaft to prevent complete removal of the bone punch. By positioning the punch stop on the handle, the physician is provided with good control over retraction of the bone punch, as the physician will typically grasp the handle with one hand while pulling back (proximally) on the bone punch handle to retract the bone punch with the other hand. The anchor tube 20 can be seen to align with the open slot 86, illustrating the path the bone punch passes along.

FIGS. 5A-5D show section views of a portion of an example implantation tool in first and second configurations. Starting in FIG. 5A, the section view is at the direction indicated at line 5A-5D in FIG. 1. The inner workings of the magazine 60 can be seen. The plurality of cartridges 70 are pushed in a distal direction by a spring 64 using a plunger 62. As each cartridge 70 is used and removed, the plunger 62 advances distally under force applied by the spring 64. The spring force maintains the distalmost of the cartridges pressed against the magazine front stop 250.

The punch stop 87 can also be observed. In FIG. 5A, the punch stop 87 is pushing against a smooth part of the bone punch shaft 46 and rests, under spring pressure, in a disengaged position. The lower slide 32 in this section view can be observed to include the trigger link slot 152 and lower slide slot 160, which is in alignment at its distal end with the distal end of the cartridge opening 110 in the upper slide 26.

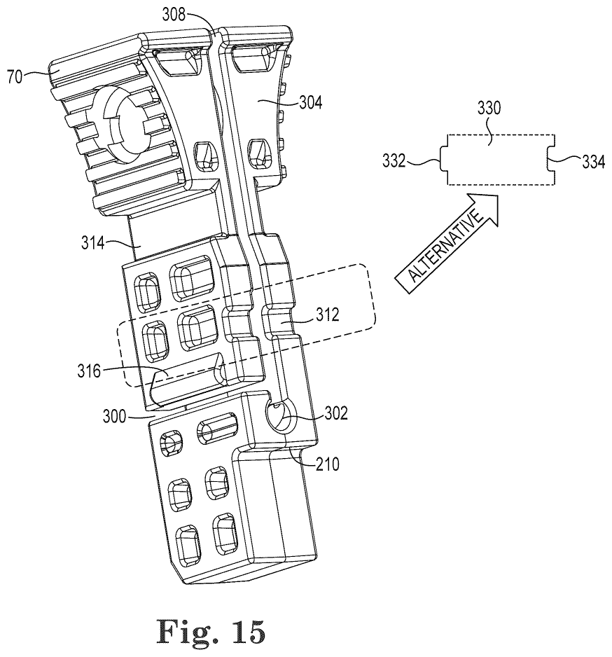

The detail view in FIG. 5B corresponds to the box shown in FIG. 5A. Here, each cartridge 70 can be seen to include a shoulder 210. The magazine front stop 250 may include a cartridge retainer 256 (further shown in FIG. 12B) that engages with a cartridge indent 312 (further shown in FIG. 12C) to hold the distalmost cartridge 70a in the “up” position shown in FIGS. 5A-5B until the physician desires to push the cartridge down to a position shown in FIGS. 5C-5D. Alternatively, the cartridge retainer 256 may be omitted from the magazine front stop 250. In some examples, no cartridge retainer function is needed. In other examples, each cartridge may include a design as shown at 330 in FIG. 15 (as explained in reference to FIG. 15), so that one cartridge engages the next in line when held in an up position.

The lower slide 32 generally aligns with the upper slide 26 at an offset. As shown, the lower slide slot 160 is offset at its distal end relative to the cartridge opening 110. The cartridge opening 110 is defined on its distal end with a ledge 112, positioned to engage with the shoulder 210 on each cartridge to limit downward movement of the cartridge 70a when the physician elects to depress the cartridge 70a. It should be noted that the position of the bone punch shaft 46 prevents the cartridge 70a from being depressed into the cartridge opening 110 and lower slide slot 160; to depress the cartridge 70a, the bone punch must first be retracted in the proximal direction.

FIG. 5C shows a next step in the anchor implantation procedure. Here, the bone punch shaft 46 has been retracted to its fullest extent. The punch stop 87 drops down under spring pressure to engage an indent or notch or other feature, such as an indented ring, on the bone punch shaft and prevents complete removal of the bone punch from the implantation tool. Because the punch stop is secured to the housing, the step of withdrawing the bone punch does not affect the relative positioning of the upper slide 26, which carries the magazine 60/and cartridges. Further, the initial extraction of the bone punch is performed with the housing and, in particular, the distalmost cartridge, in the configuration shown in FIGS. 5A-5B. Thus, when the trigger is actuated to pull the lower slide proximally, which in turn pulls the bone punch proximally, the upper slide stays in place. The upper slide latch, as previously described, is frictionally engaged with the upper slide to reduce unwanted movement. In addition, friction between the inner tube and the formed bone hole, all work to keep the inner tube and upper slide from retracting in the proximal direction when the trigger is actuated to remove the bone punch from the bone hole.

Having retracted the bone punch proximally, the physician next delivers an anchor to the patient. Anchor delivery starts with depressing the distalmost cartridge 70A into the cartridge opening 110 of the upper slide, and continuing to depress the cartridge until the shoulder 210 of the cartridge 70a abuts the ledge 112, as shown in FIG. 5D. A protrusion 316 on the cartridge will also engage the side opening 114 of the upper slide, though this is not visible in FIG. 5D. When the cartridge 70a is fully depressed as shown in FIG. 5D, the anchor space 302 (and thus anchor 400) are aligned with the first lumen 102 and second lumen 104 of the upper slide 26. As a result, once positioned as shown in FIG. 5C/5D, advancing the bone punch from the first lumen 102, into the anchor space 302 will push the anchor 400 in the distal direction, into the second lumen 104 and then into the inner lumen of the inner tube. In some examples, this position of the cartridge that places the anchor space 302 into alignment with the inner lumen of the inner tube (along with the first and second lumens) can be referred to as a delivery position or anchor delivery position for the cartridge.

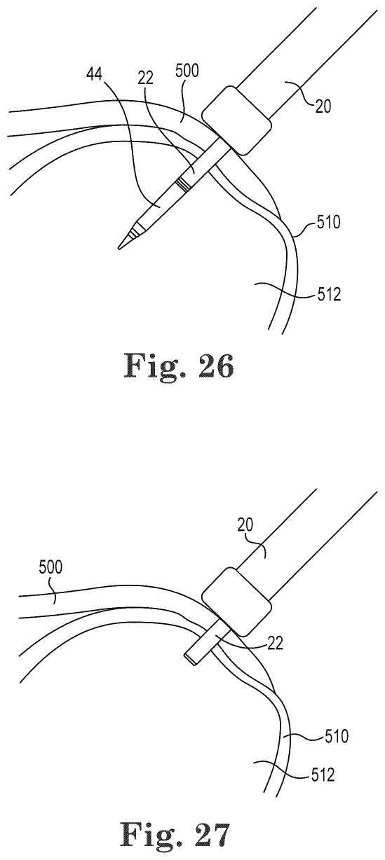

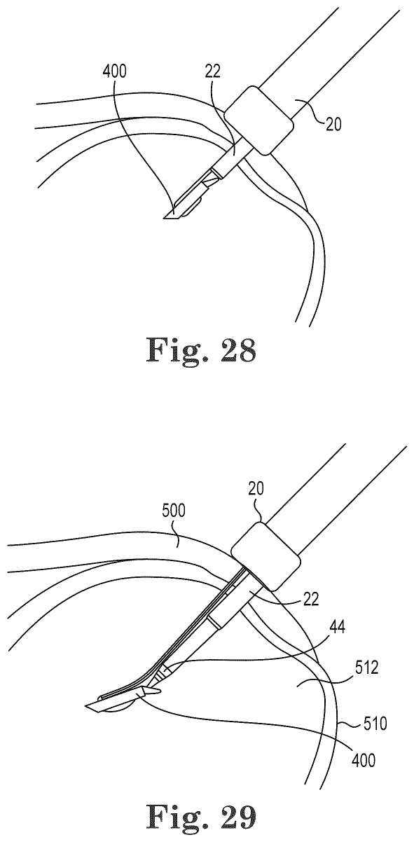

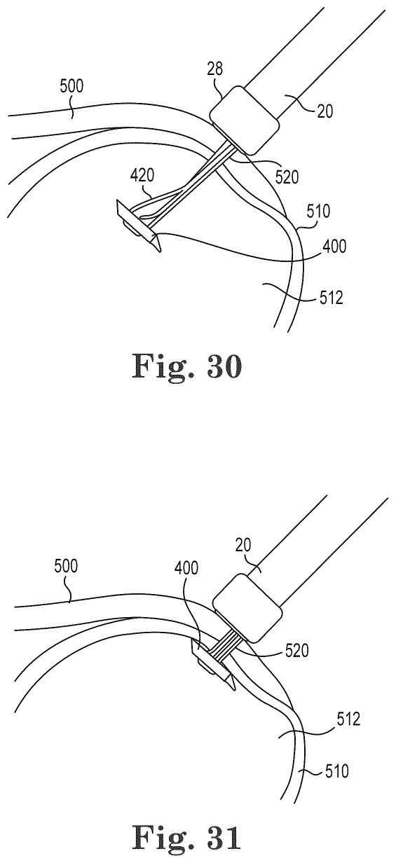

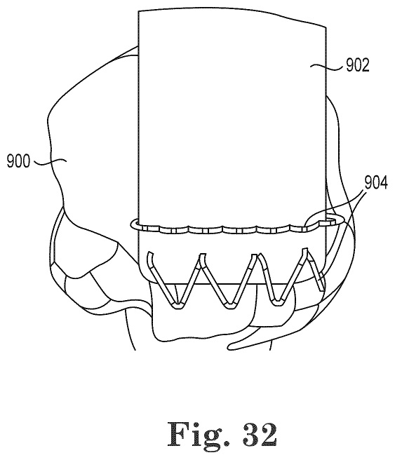

The physician will advance the anchor by pushing with the bone punch until the anchor is positioned in the bone hole. As this occurs, the physician will receive tactile feedback as the bone punch latches meet the proximal, middle and distal detents shown in FIG. 4A. In some embodiments more or less tactile feedback positions can be included. After fully inserting the anchor, the physician will then want to retract the bone punch as well as the inner tube. To do so, the physician again actuates the trigger. This time, however, the position of the cartridge in FIGS. 5C and 5D extends into the lower slide. With the cartridge 70a in the fully depressed position, or anchor delivery position, when the trigger is actuated, the force applied will retract the lower slide and the distal end of the lower slide slot 160 will push against the distal side of the cartridge where it extends into the lower slide slot 160. It should be noted that the offset of the distal end of the lower slide slot 160 from the cartridge opening 110 means that the initial movement will first release the bone punch latch (See FIGS. 11A-11C) from the housing before applying force to the cartridge and linked upper slide and magazine. Once the initial offset is passed, the retraction force is communicated to the upper slide by the cartridge, causing the upper slide 32 and the attached inner tube 22 to also retract. Retracting the inner tube 22 after the anchor is placed reduces any likelihood that the sutures at the anchor will be nicked or cut by the distal end of the inner tube 22 during manipulation of the position of the anchor (see FIGS. 26D-26G). User mistakes in the order of steps are also discouraged by this sequence, as the cartridge 70a cannot be removed from the anchor space until at least the bone punch is retracted until the distal tip of the bone punch is proximal of the anchor space 302 in the cartridge 70a.

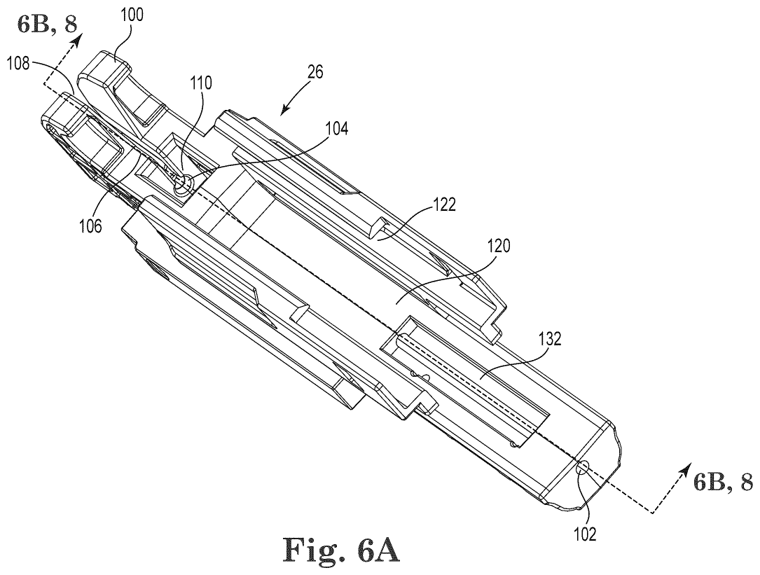

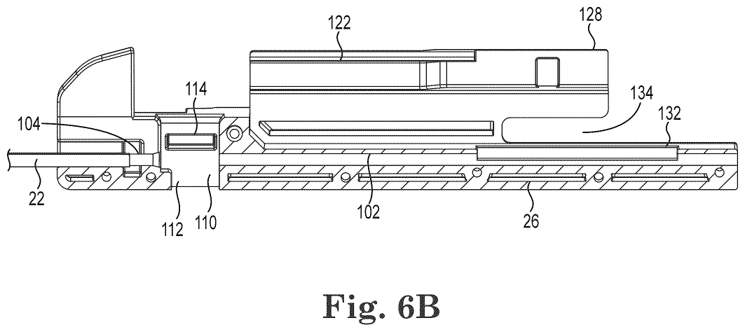

FIGS. 6A-6B are perspective and section views of the upper slide. Starting with FIG. 6A, the upper slide 26 has a front stop 100 against which the distal end of the magazine will rest when fully inserted to the upper slide 26. The upper slide 26 defines a first lumen 102 that extends from the proximal end of the upper slide 26 to the cartridge opening at 110. A portion of the first lumen 102 is open along a top side thereof, as shown at 132, allowing interaction of the punch stop 87 with the bone punch shaft 46 therethrough.

The upper slide 26 also has a second lumen 102 that extends from the cartridge opening at 110 to the distal end of the upper slide 26. Though not shown in the isolation view of FIG. 6A, the inner tube is secured in the second lumen 104. The first and second lumens align with one another so that the bone punch can be passed into and through the first lumen 104, to and then through the cartridge opening 110, and then into the second lumen 104 and inner tube (not shown). The second lumen 104 has a top slot 106 which aligns with a suture slot 108 at the distal end of the upper slide.

The cartridge opening 110 is sized to receive a cartridge as shown in FIGS. 5C-5D. A ledge 112 is provided in the cartridge opening 110 of the upper slide 26. The ledge 112 provides a surface against which a corresponding shoulder on the cartridge comes to rest when fully inserted into the cartridge opening 110, allowing a lower portion of the cartridge to extend still further down into the lower slide.

The upper slide 26 is also adapted to receive the magazine 60 that holds the cartridges 70 (FIGS. 1-2). A magazine receiving area is shown at 120. A magazine receiving channel 122 receives elongated tabs on the magazine, which slides with the tabs under compression forces into the magazine receiving area 120 until abutting the front stop 100. On the outside of the upper slide is a handle channel 126 that engages a corresponding shelf of the handle/housing, allowing sliding therebetween.

The section view of FIG. 6B is taken along line 6B, 8 in FIG. 6A. The section view illustrates how the upper slide 26 includes the first lumen 102, with the opening 132 (which may be referred to as a punch stop slot). A gap 134 is visible toward the proximal end. The gap 134 allows the upper slide 26 to move relative to the housing/handle without interfering with the punch stop (FIG. 4B). The magazine receiving channel 122 includes a magazine holder 128, which is positioned to receive a tab on the magazine to thereby hold the magazine in a fully inserted position.

FIG. 6B also provides another view of the location of the cartridge opening 110. The ledge 112 is at the distal side of the cartridge opening 110, and limits downward movement of the cartridge when inserted therethrough, as shown in FIGS. 5C-5D. The side opening 114 is arranged so that when a cartridge is fully inserted into and through the cartridge opening 110, the protrusion 316 (FIG. 12D) slides into the side opening 114 and requires force to remove the cartridge in the upward direction.

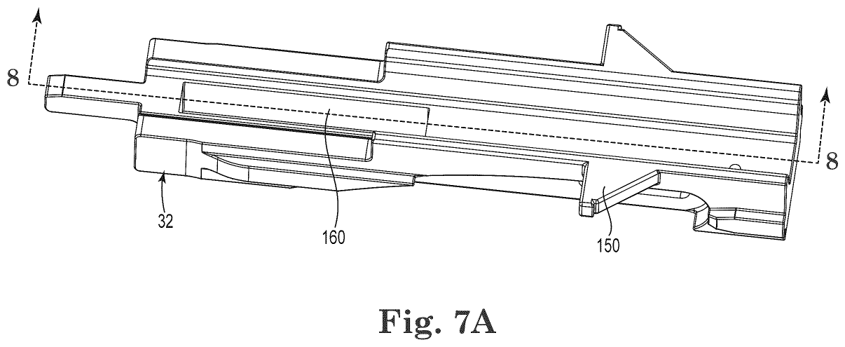

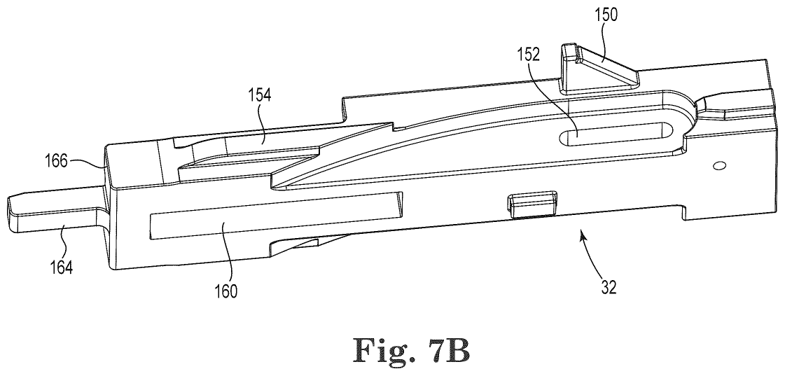

FIGS. 7A-7B show upper and lower perspective views of a lower slide 32. The view from above is shown in FIG. 7A, with a flat area at 160 which is the lower slide receiving are. The bone punch release tab can be seen at 150.

The view of lower slide 32 from below in FIG. 7B shows how the trigger link slot 152 is positioned. A handle engaging ridge 154 is included and provides smooth sliding relative to the handle/housing. A lower slide abutment is shown at 166, and is split by a lower slide extension shown at 164. The fork in the trigger will go on either side of the lower slide extension 164, as highlighted in FIGS. 11A-11C.

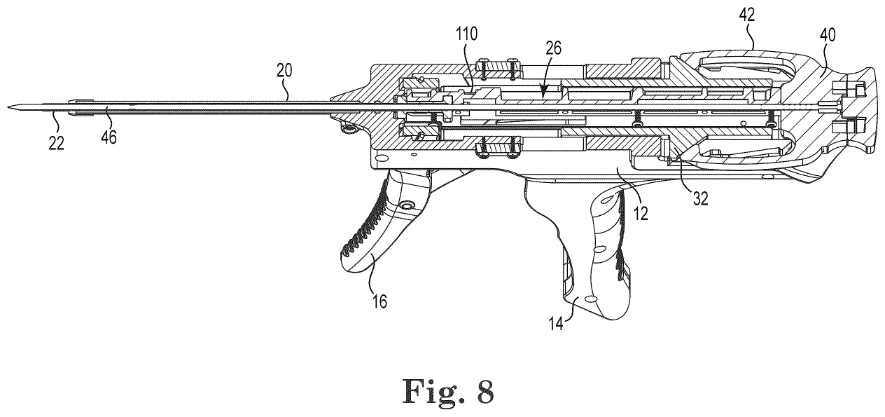

FIG. 8 is a top-down section view of an example implantation tool. Here, the bone punch 40 is fully inserted, so that the bone punch latch 52 is engaged with the distal detent (item 82 in FIG. 4A). The lower slide 32, with the bone punch release tabs 150 can be seen; in the fully inserted position of the bone punch 40, the bone punch release tabs 150 are adjacent the arms of the bone punch latch 42. The cartridge opening can be seen at 110, near the distal end of the upper slide 26. The anchor tube 20 is attached at the distal end of the housing 12, and the inner tube 22 extends to and is attached within the upper slide 26. The bone punch shaft 46 can be seen crossing the cartridge opening 110 as well, and extends past the distal ends of both the anchor tube 20 and the inner tube 22.

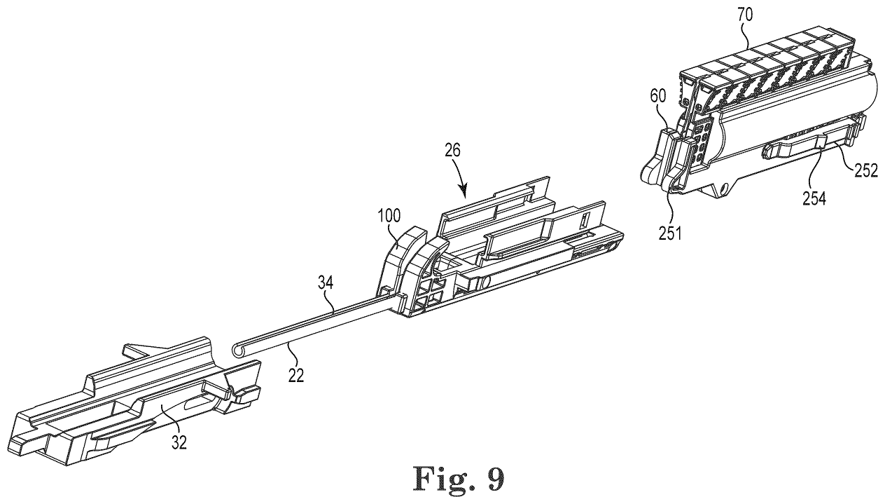

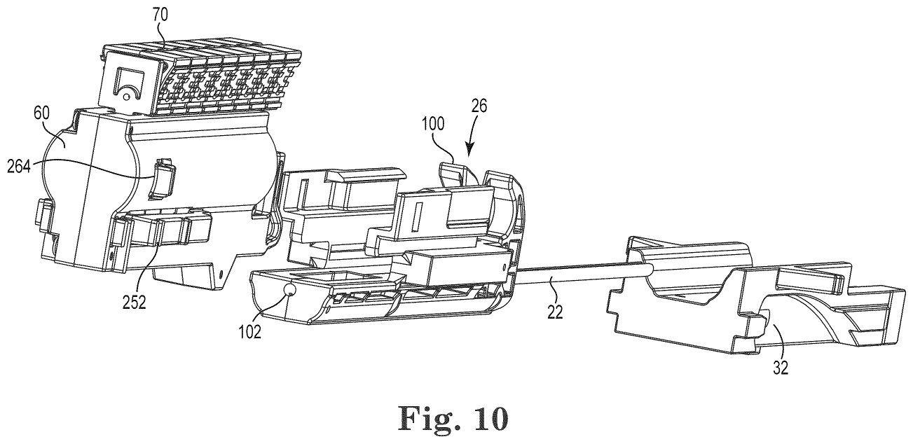

FIGS. 9 and 10 are front and rear partial exploded views of a magazine, upper slide and lower slide. In this disassembled state, the lower slide 32 can be seen in its relative position to the upper slide 26. Further, the cartridge 60 is shown aligned with the upper slide 26, carrying several cartridges 70. Along the side of the magazine 60 are magazine tabs 254, with a magazine retainer 252 thereon. To place a magazine 60 relative to the upper slide 26, the magazine 60 is slid along the top of the upper slide with the magazine tabs 254 aligned with the magazine receiving channel 122. This compresses the magazine retainers 252. As the magazine 60 is advanced, the magazine tabs 254 will snap into the magazine holder 128 (FIG. 8), at the same time as the magazine front 251 engages the proximal side of the front stop 100 of the upper slide 26. To remove the magazine 60, the proximal ends of the magazine retainers 252 are squeezed toward one another, releasing the magazine tabs 254 from the magazine holder 128 (FIG. 8).

FIGS. 11A-11C show an illustrative process of actuating the trigger. In FIG. 11A, the trigger is in its resting position, pushed distally by the trigger springs 21 (FIG. 4B) contained in the housing. The lower slide 32 is shown in its distalmost position, which would correspond to, for example, the bone punch being fully inserted at its most distal position, with the bone punch latch secured to the distal detent 82 (FIG. 4A). It should be noted that the distalmost position for the lower slide could be achieved manually without the bone punch being in its distalmost position.

The physician at this point begins to pull the trigger 16 toward the handle, that is, in a proximal direction, as shown. The trigger link 18 is coupled to the trigger link slot 152 using the link pin 153. However, during the initial pull, the link pin 153 is at the distal end of the trigger link slot 152 and applies no force on the lower slide, instead sliding within the trigger link slot 152 in the proximal direction. Instead, a trigger abutment 19 presses against the lower slide abutment 166, causing the lower slide 32 to move in the proximal direction. The geometry used in this initial portion of the trigger pull allows the force applied at the lower end of the trigger 16 to be multiplied. That is, the trigger operates as a second class lever. The mechanical advantage is therefore the length from the trigger pivot 17 to the location on the trigger where force is applied, to the length from the trigger pivot 17 to the trigger abutment. In the example shown, the mechanical advantage, depending on finger placement, may be in the range of about 2:1 to about 4:1.

Turning next to FIG. 11B, the actuation of the trigger in the proximal direction continues. The lower slide 32 continues to move in the proximal direction and the trigger link 18 moves the link pin 153 toward the proximal end of the trigger link slot 152. Once the link pin 153 reaches the proximal end of the trigger link slot, as shown in FIG. 11C, the trigger link 18 begins to push the lower slide 32 in the proximal direction. At this point, the mechanical advantage, again depending on finger placement, is in the range of about 1:1 to about 1.5:1. The design thus provides greater mechanical advantage when the trigger is first actuated, as follows:

If the cartridge is in a position as shown in FIGS. 5A-5B, the lower slide and bone punch are drawn in the proximal direction. The increased mechanical advantage during the first part of the trigger pull will aid in releasing the bone punch from the bone hole. Once the bone punch is released (it may still enter the bone hole but to a reduced extent), the force needed to retract the bone punch is diminished. The upper slide and attached inner tube are preferably not moved by retraction of the bone punch and lower slide when the cartridge is not depressed.

If the cartridge is in a position as shown in FIGS. 5C-5D, as the lower slide is pulled proximally (along with the bone punch), the distal end of the lower slide slot contacts the cartridge and pushes the cartridge in the proximal direction, transferring force to the upper slide and magazine. Because the distal end of the inner tube would be engaged with the bone hole, the added mechanical advantage aids in releasing the inner tube from the bone hole. Once the inner tube is released, the needed force for further proximal movement is reduced.

Thus as shown in FIGS. 11A-11C, the trigger has a trigger range of motion. During the first portion of trigger action through the trigger range of motion, the interaction of the abutment 19 with the distal end of the lower slide, that is, lower slide abutment 166, causes retraction in a proximal direction of the lower slide. It should be noted that the bone punch release tab 150 push against the bone punch latch 42 throughout the range of motion to both spread the bone punch latch to release from the housing detent(s), and to push the bone punch in the proximal direction. Thus this initial movement in the trigger range of motion retracts the bone punch from the bone hole by the action of the bone punch release tab 150 against the bone punch latch 42. Later portions of the trigger range of motion, as in FIG. 11C, rely on the trigger link to push the lower slide 32 and bone punch release tab 150 in the proximal direction. In some examples, the bone punch handle does not in fact come into contact with the proximal end of the lower slide during retraction; in other examples, the proximal end of the lower slide may press against the bone punch handle.

FIGS. 12A-12D show a rear perspective and three cut-away views of the magazine with cartridges. Starting with FIG. 12A, the magazine 60 is shown from a proximal or rear side. The magazine retainer 252 extends from the proximal end toward the distal end, about half of the length of the magazine (though this is not limiting). The magazine tabs 254 extend further out from the magazine retainer 252. The proximal end of the magazine retainer 252 is free, and can be flexed inward to allow release of the magazine tabs 254 from the upper slide. The positioning of the plunger 62 against the last or most proximal cartridge is observed as well.

At the distal end of the magazine 60 is the magazine front stop 250, which is used to stop the distal movement of the cartridges under force applied via the plunger 62 and spring (not shown). Here, the distal or first cartridge 70a is shown in the depressed position, with a top of the first cartridge 70a approximately flush with the top of the magazine front stop 250. Magazine arms 258 extend along either side of the first cartridge 70a.

FIG. 12B is a partial cut-away view along line B-B of FIG. 12A. The magazine arms 258 are shown in section, and define a magazine opening 260 therebetween. When the magazine is fully inserted into the upper slide, the magazine opening 260 will line up with the cartridge opening of the upper slide. The magazine front stop 250 includes a suture gap 259 between the distal ends of the two magazine arms, allowing management of the sutures that are engaged with the anchor in each cartridge. A cartridge retainer 256 projects in the proximal direction from the magazine front stop's 250 proximal side, and is positioned and sized to engage with a corresponding cartridge indent on each cartridge, thus defining an upper position in which the cartridge can be held, preventing the cartridge from sliding up or down without the application of an external force. Alternatively, the indent or projection can be included on the proximal side of a front cartridge and interact with indent or projection on the distal side of the next cartridge to prevent the distalmost cartridge from sliding up or down without the application of an external force, as shown in FIG. 15 at 330.

FIG. 12C is a partial cut-away view along line C-C of FIG. 12A. Here, the magazine can be seen, including the placement and illustrative design for the magazine retainer. A suture cleat 264 is shown on the magazine as well. When the assembly of the magazine 60 and cartridges 70 is complete, a portion of a working suture that extends through all the anchors (at least in some examples) is hooked around the suture cleat to hold the suture in a desirable position, maintaining light tension on the sutures and anchors to prevent tangling of the suture. Again, the magazine arms extend distally from the magazine 60, defining a magazine opening 260.

FIG. 12D provides another view, straight on facing proximally at the first cartridge. The magazine 60 holds the cartridge 70, with a cartridge neck 314 passing within an upper slot of the magazine 60. The spool is positioned above the cartridge neck 314. The cartridge 70 includes a shoulder 210, which can be used to abut a ledge in the upper slide and thereby define a maximum depth of depressing the cartridge 70. The anchor space is shown at 302, and contains an anchor 400. A working suture 420 extends from the anchor 400, up toward a front notch 308 and over the top of the cartridge to the next subsequent cartridge which is held in the magazine. The anchor space 302 is defined by the cartridge 70 with sidewalls that may be generally cylindrical, though a polygonal shape or combination of rounded and straight walls can be used as desired. The anchor space 302 is open along the length thereof at the top or upper portion, allowing each of a suture lock and a working suture 420 to pass therethrough. The anchor space 302 is open at both proximal and distal ends.

The cartridge upper portion 304 defines the front notch 308, and includes on the sides thereof grips for aiding a physician or other user to grasp the upper portion 304 for purposes of removing the cartridge from the cartridge opening 260 and the rest of the implantation tool. The cartridge also houses a spool 306, which is used in some examples to hold a suture lock.

FIG. 12D also shows how the protrusion 316 is positioned and extends into the magazine channel 262, thereby also defining an upper position for the cartridge. When the cartridge is depressed into the housing to position the anchor space 302 an anchor 400 in alignment with the lumens of the upper slide and the bone punch, the protrusion 316 engages with the side opening 114 (FIG. 8) of the upper slide. The protrusion 316 flexes inward as allowed by the cartridge gap shown at 300. The anchor 400 is protected on its lateral sides by the anchor space 302, which is generally cylindrical, though it may have flattened bottom or sides as desired, preventing rotation of the anchor as it is held in position. The anchor space is sized to retain the anchor and allow controlled directional passage of the anchor distally from its position in the anchor space into the proximal end of the inner tube when the distal tip of the punch pin pushes the anchor forward. The anchor space is open at proximal and distal ends.

FIG. 12E illustrates, from a front (or distal end) view, positioning of the front cartridge 70a and subsequent cartridge 70b. The front cartridge 70a is in its fully depressed position, ready for delivery of the anchor contained in the anchor space 302, with an upper end flush with the magazine front stop 250. As to the subsequent cartridge 70b, the anchor delivery space distal end is blocked by the upper portion of the front cartridge 70a, protecting the anchor in the subsequent cartridge 70b from being dislodged. In this way, the anchors in each subsequent cartridge 70b are protected throughout the procedure of implanting the anchors. Once a cartridge is advanced to the front or distalmost position, the anchor space is protected by the magazine front stop until pushed down into the delivery position.

FIG. 12F illustrates certain features of the implantation tool, using a rear elevation view with the bone punch entirely removed. With the bone punch omitted, it can be seen how the housing holds the lower slide 32, which in turn holds the upper slide 26. The magazine 60 rests on top of the upper slide 26. The bone punch release tabs 150 of the lower slide can be seen extending out of the open slots (see open slots 86 of FIG. 4A) in the housing. The width of the housing 12, shown at 12w, exceeds the width of the magazine 60, shown at 60w. This is useful to the extent the physician is able to see around the magazine while manipulating the tool. The magazine 60 can also be seen to be centrally positioned in relation to the housing, so that it is not necessary for the physician to twist or turn the implantation tool to see around the magazine, making for an ambidextrous tool that can be used equally on left and right sides of the body and/or by left-handed and right-handed physicians.

As highlighted above and below, the magazine and/or cartridges may contain features that retain the cartridges in the magazine, including with spring pressure and friction (the magazine spring, as shown in FIGS. 5A and 5C) as well as the indents and retention or projection features shown in FIG. 15. This means that during use, the physican is able to hold and/or turn the implantation tool at various angles without worry that a cartridge will fall out. Moreover, the anchor(s) are held within the cartridges, with the ends of the anchor space covered/blocked except when it is desired to actually remove an anchor by passing the bone punch therethrough.

FIGS. 13-16 illustrate the cartridge with an anchor therein. Starting at FIG. 13, the cartridge 70 is shown including the cartridge indent 312. A front notch 308 faces the distal direction in use (as defined in FIG. 1). The front notch 308 assists in managing the position of a working suture that passes from one anchor to the next in an array. The shoulder 210 can be seen on the front or distal side of the cartridge 70. The shoulder 210 is used to limit the downward movement of the cartridge when pushed down until it abuts the ledge 112 of the upper slide 26 as seen in FIGS. 5C-5D.

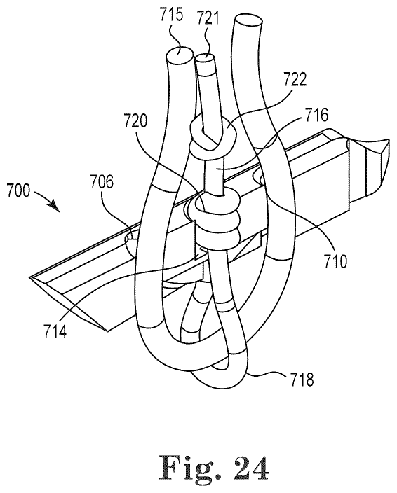

In some examples, a suture lock is provided for the anchor (see FIG. 24), and has a first end which wraps about or encircles a working suture adjacent the anchor. The suture lock may include an intermediate portion that passes through a bore of the anchor. The second end of the suture lock is managed internal to the cartridge by including a spool 306 in the upper portion 304 of the cartridge 70. The spool 306 has slots and indentations as shown that allow engagement with a tool for winding the spool 306 to hold the suture lock.

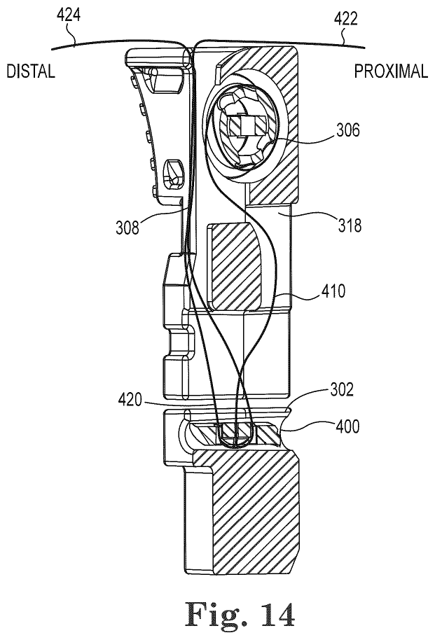

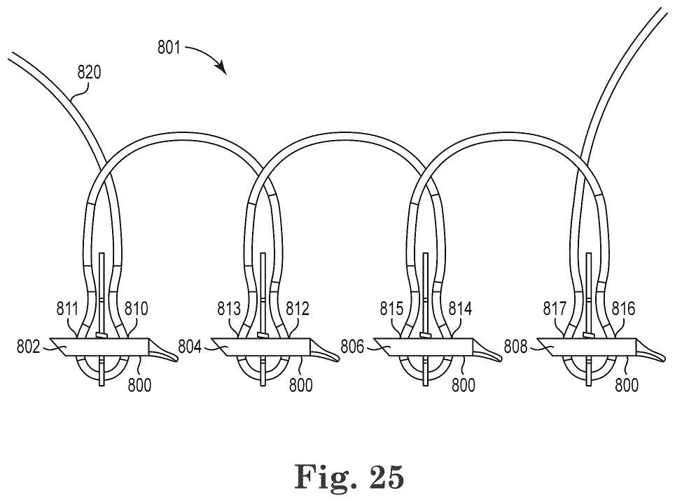

FIG. 14 is a cut-away view of the cartridge and its contents along line 14 -14 of FIG. 13. The anchor 400 is shown held in the anchor space 302 of the cartridge, with a working suture 420 shown passing into a first bore from a top side of the anchor 400, then along the bottom side of the anchor 400 and up through a second bore of the anchor 400. The anchor 400 may be part of an anchor array as shown in FIG. 25, with a single working suture 420 coupled to each anchor in the array. The working suture 420 then passes up along the front notch 308 and over the top of the cartridge 70, with one end 422 passing in the proximal direction to a subsequent anchor in the array, and the other end 424 passing in the distal direction to the previous anchor in the array.

The suture lock 410 passes through the middle bore of the anchor 400 and wraps about or encircles the working suture 420 at the bottom side of the anchor 400. The suture lock 410 then extends upward, to the rear side of the cartridge and through channel 318 to the spool 306. The suture lock 410 wraps around the spool with a length of 20 to 30 cm, or more or less, so that the anchor can be pushed down the inner tube toward and then into a bone hole while the suture lock 410 unspools with the first end of the suture lock 410 remaining connected to the spool 306. The cross-hatched portions of FIG. 14 indicate the parts of the cartridge which connect the halves thereof. This allows the channel 318 to be defined as shown, separating the suture lock 410 from the working suture 420.

The suture lock 410 and working suture 420 are omitted from FIGS. 13, 15 and 16, but may be present using a similar routing as shown by FIG. 14. In other examples, different anchor designs may be used. For example, an anchor with only one bore may have the working suture 420 pass therethrough, with a suture lock 410 positioned near the anchor. In another example, a suture lock may pass through one bore and the working suture may pass through another bore of a two-bore anchor. A two-bore anchor may have the working suture pass through each bore, from top to bottom, then along the bottom and back up to the top, but omits the middle bore shown in FIG. 14, with the suture lock encircling the portion of the working suture that runs along the bottom of the anchor between the two bores. Other suitable designs can be used as desired.

FIG. 15 shows another view of the cartridge 70, with the front notch 308 for passing the working suture there-along in the cartridge upper portion 304. A cartridge neck 318 is sized to sit within the upper or top channel of the magazine 60 as shown by FIG. 12D. The illustrative position and shape of the indent 312 can be observed, with the indent 312 used to maintain the cartridge 70 in an upper position while held by the magazine 60 and pressed against the magazine front stop 250, engaging the cartridge retainer 256 as in FIGS. 5A-5B. The cartridge gap 300 allows flexing of the sidewall of the cartridge holding the protrusion 316, which is used to secure the cartridge 70 when in a depressed position to align the anchor space 302 with the lumens 102, 104 of the upper slide by engaging the side opening of the upper slide, as shown in FIGS. 5C-5D). When the protrusion 316 is engaged with the side opening 114 of the upper slide, the shoulder 210 would rest against the ledge 112 of the upper slide, as also shown in FIGS. 5C-5D.

The indent 312 may be configured to engage with the front stop of the magazine in some examples, but may be positioned or used differently in others. As shown at 330, an alternative design may position an indent 334 and a projection 332 on opposing proximal and distal sides of the cartridge. In one example, the indent 334 is on the distal side, so that no gap appears when the cartridge is pressed against the magazine front stop. The projection 332 can then be on the proximal side of the cartridge. As a result, each cartridge would be engaged with a subsequent cartridge. This design can avoid tangling or catching of the working suture and/or suture lock with a projection on the magazine front stop during use, and limits the possibility of the working suture or suture lock becoming mis-directed into a gap between the cartridge and the front stop of the magazine. In other examples, the indent 334 can be on the proximal side and the projection 332 on the distal side of the cartridge. The indent 334 or projection 332 would also have a counterpart structure on the plunger of the magazine to manage the most-proximal or last cartridge.

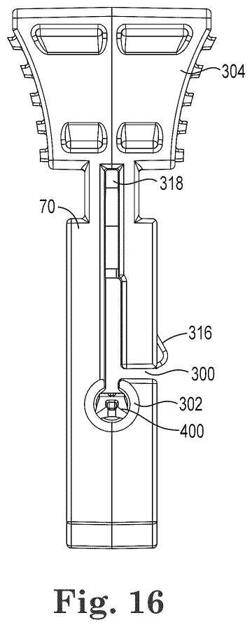

FIG. 16 is a rear elevation view of the cartridge 70, with the cartridge upper portion 304 having ridges thereon which may aid when grasping the cartridge to remove it from the magazine after use, for example. The channel 318 can be observed as creating a through-hole or opening. The protrusion 316 and cartridge gap 300 are also illustrated, and anchor 400 is in the anchor space 302.

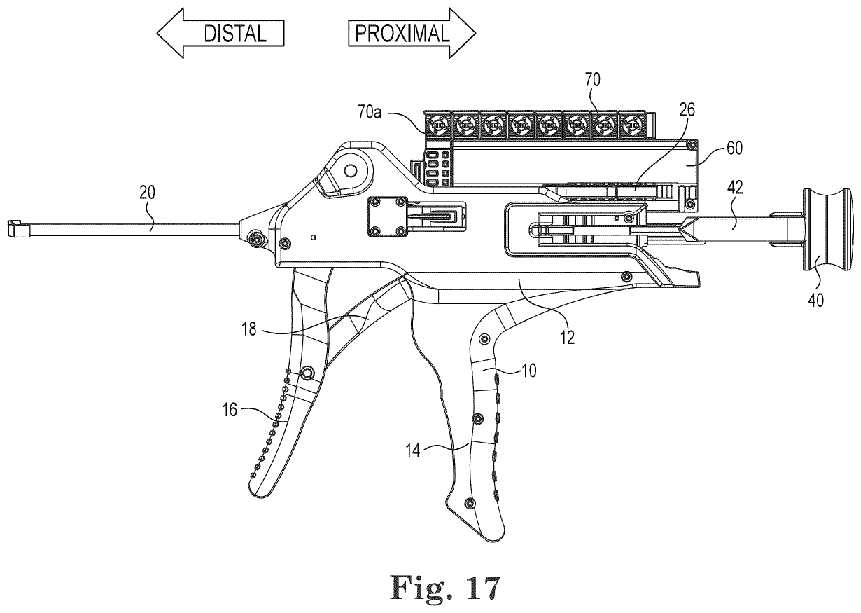

FIGS. 17-22 illustrate a step by step process of using the implant tool. FIG. 17 shows the implant tool 10 prior to placement of an anchor. The first cartridge 70a in the magazine 60 is against the front stop of the magazine 60, with the magazine engaged with the upper slide 26. The upper slide 26 is freely moveable in relation to the lower slide 32, (with designed frictional resistance of the upper slide relative to the housing) which is itself moveable relative to the housing 12 of the implant tool 10, subject to limitation by the trigger link 18 that is attached thereto. The lower slide 32 is biased in the distal direction by the trigger spring (FIG. 4B). The bone punch 40 is inserted into at least the first lumen of the upper slide, but is not latched to the housing. The terms distal and proximal are used consistent with the arrows in FIG. 25, and this terminology will be used in similar fashion for the remaining figures.

With the device 10 as shown in FIG. 17, and with the patient prepared (sterile field, and access to a tendon or other tissue that is to be attached to a bone established), the physician grasps the handle 14, likely also holding the trigger 16 without depressing or actuating the trigger, and positions the anchor tube 20 at a desired location. For example, for repair of a torn rotator cuff, a tendon is positioned against bone, preferably in the original footprint of the tendon on the bone. The distal end of the anchor tube 20 is pressed against the tendon. The physician will then advance the bone punch 40 into the tool 10 until the distal tip of the bone punch exits the distal end of the anchor tube. As the bone punch 40 is advanced, the bone punch latch 42 will engage at least the proximal detent (FIG. 4A) on the housing. As the distal tip of the bone punch 40 exits the distal end of the anchor tube 20, it will start to pierce the tendon or other tissue that is to be secured to the bone.

Alternatively, the physician may advance the bone punch before pressing against the tendon, and may, for example, advance the bone punch to engage the bone punch latch 42 with the middle detent (FIG. 4A) on the housing, and uses the distal tip of the bone punch as a probe to check the quality, hardness and/or stability of the bone to select a location for an anchor to be implanted. With a desired location selected, the physician then starts to press the handle distally to advance the bone punch into and through the tendon.

When resistance to the advancement of the bone punch builds, the physician may tap or pound against the handle of the bone punch 40 to pierce through the tendon and establish a bone hole, with or without using a mallet, as desired. As pressure is applied in the distal direction, the focus is on advancing the bone punch relative to the housing 12, and so the bone punch latches 42 will engage with the distal detent (FIG. 4A) on the housing 12, becoming latched thereto. As this occurs, the upper slide is pushed distally by the bone punch, against which it abuts as the bone punch moves distally. When the bone punch is fully advanced in relation to the housing, the upper slide will also be in its distalmost position relative to the housing, which will advance the inner tube beyond the distal end of the anchor tube 20. Alternatively, the bone punch and upper slide can be advance to their distalmost position prior to insertion into the tendon.

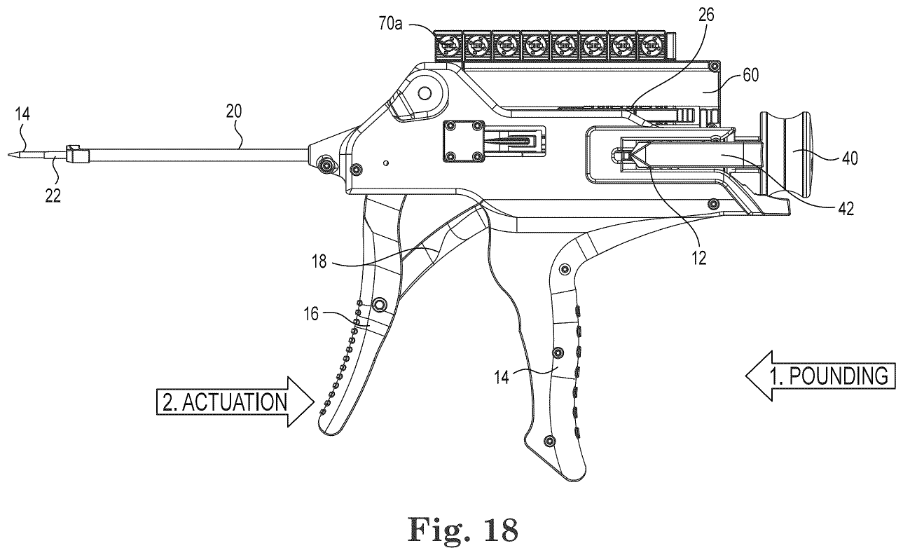

When the bone punch 42 is fully advanced in relation to the housing 12, the tool 10 will have the configuration shown in FIG. 18. The bone punch latch 42 is engaged with the distal detent (FIG. 4A) on the housing 12, and the bone punch 40 is in a distal-most position. The bone punch handle abuts the proximal end of the upper slide 26. In this position, the inner tube 22 has been pushed distally, along with the upper slide 26, relative to the anchor tube 20 which is fixed to the housing 12. The bone punch tip 44 extends beyond the distal end of the inner tube and the distal end of the anchor tube. In this configuration, the physician will continue advancing the bone punch tip 44 into the bone hold until a desired depth is achieved. Often the depth desired will position the distal end of the anchor tube 20 firmly against the outside of the tendon with the inner tube 22 passing through the tendon and into the bone hole.

FIG. 26 illustrates the status at the distal tip. Here, the bone punch tip 44 has been pounded, tapped or otherwise advanced through a tendon 500, which may be the supraspinatus tendon in some examples. If desired, other tendons and/or other tissue (ligament, meniscus, etc.) and/or artificial replacement tissue can be positioned and anchored if desired. The continued advancement of the bone punch tip 44 also pierces the cortical shell 510 of the bone, which may be the humeral head or any other desired bone, and has entered the cancellous bone 512 beneath the cortical shell 510. The upper slide moves as well with the bone punch for at least a portion of this extension, so that the inner tube 22 (attached to the upper slide) extends through the tendon 500 and cortical shell 510, preferably into the cancellous bone 512. The bone punch tip 44 may extend, for example and without limitation, about 1 to about 4 cm beyond the distal end of the anchor tube 20, and the inner tube 22 may extend about half that distance.

Turning back to FIG. 18, pounding, tapping or otherwise advancing the bone punch 40 and bone punch tip 44 (with the inner tube 22 as well) takes place in a first, distal direction until the physician is satisfied with the bone hole. Then the physician actuates the trigger 16, as indicated. Trigger actuation uses the abutment 19 (FIGS. 11A-11C) initially to apply a relatively larger force and leverage advantage to retract the bone punch 40 and bone punch tip 44 from the bone hole, while also releasing the bone punch latch 42 from the housing. This releases the bone punch 40. The physician continues, however, to apply pressure in the distal direction using the handle 14 to maintain engagement of the anchor tube 20 with the tendon. Friction between the inner tube and the bone hole will keep the inner tube 22 in the bone hole as the bone punch 40 is retracted; added friction is provided to the upper slide with the upper slide latch 27 (FIG. 2) to limit or prevent retraction.

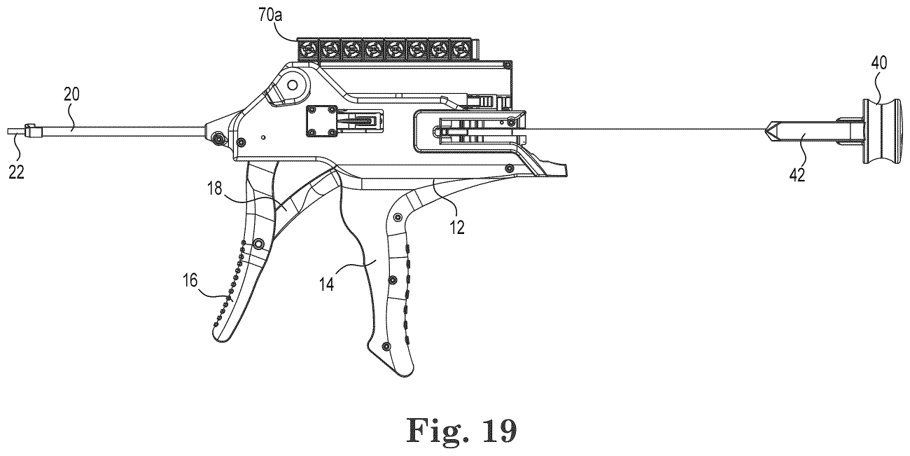

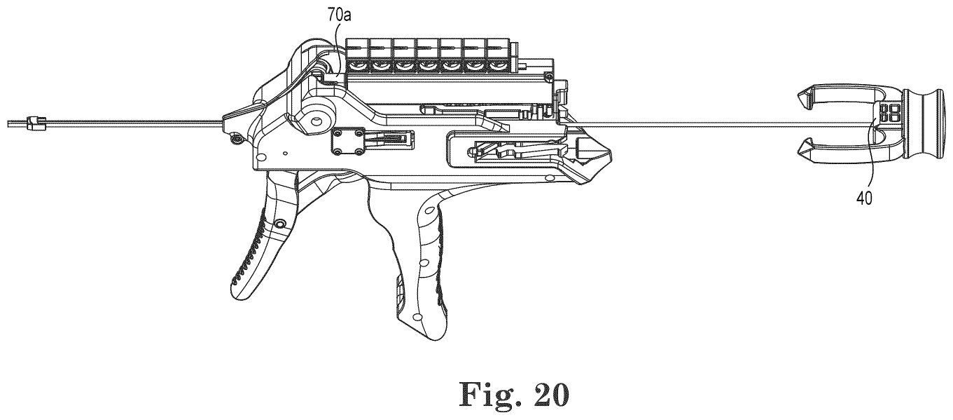

The physician will grasp the handle of the bone punch and continue retracting the bone punch until the distal tip of the bone punch is proximal of the cartridge opening 110 of the upper slide 26 (FIGS. 5A-5D), and may continue to retract the bone punch until the punch stop (FIG. 4B) is engaged. The tool 10 then has the configuration shown in FIG. 19. With the tip of the bone punch proximal of the cartridge opening, the first cartridge 70a in the magazine 60 can then be depressed down into the cartridge opening, as shown in FIG. 20. The cartridge 70a is pushed down until the protrusion 316 engages the side opening 114, and the shoulder 210 abuts the ledge 112 (FIGS. 5C-5D). This positioning of the cartridge 70a brings the anchor 400 and the anchor space 302 of the cartridge into alignment with the lumens of the upper slide 26.

At this point, the status at the bone hole is as shown in FIG. 27. The bone punch has been retracted, leaving behind the bone hole, in which the inner tube 22 is positioned. The inner tube 22 thus maintains registration with the bone hole, facilitating the next steps in which the anchor is advanced inside the inner tube 22 and into the bone hole.

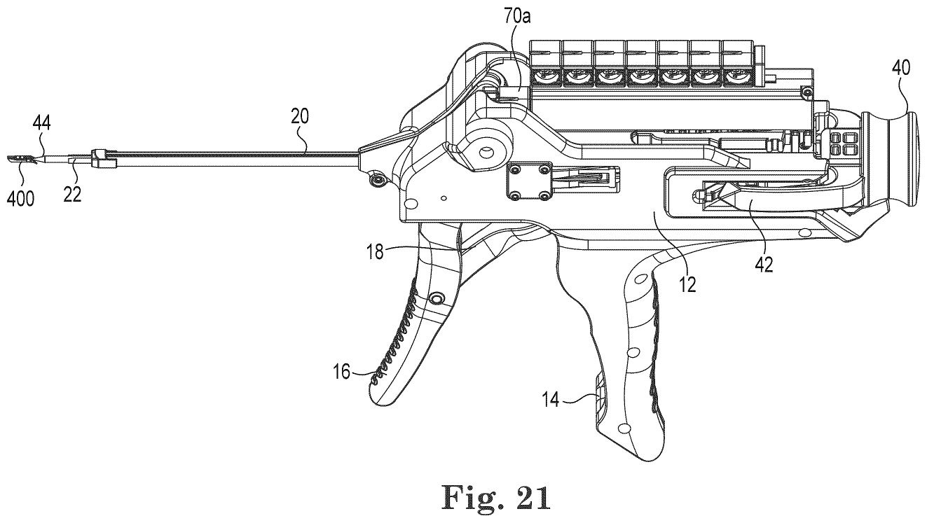

The physician next advances the bone punch 40 to push the anchor into the inner tube and down past the distal tip of the inner tube, as shown in FIG. 21. This is achieved by advancing the bone punch 40 until the bone punch latch 42 engages the distal detent of the housing 12. As the anchor is advanced, the bone punch passes through both lumens of the upper slide as well as the anchor space in the cartridge 70a.

Now, the anchor is positioned in the bone hole as shown in FIG. 28. The anchor 400 may be angled at its own proximal and/or distal ends to encourage turning of the anchor as it exits the inner tube 22, starting the toggling of the anchor toward a desired final position. This angling occurs as the anchor is released from the inner tube 22 and the bone punch continues to advance, as shown by FIG. 29.

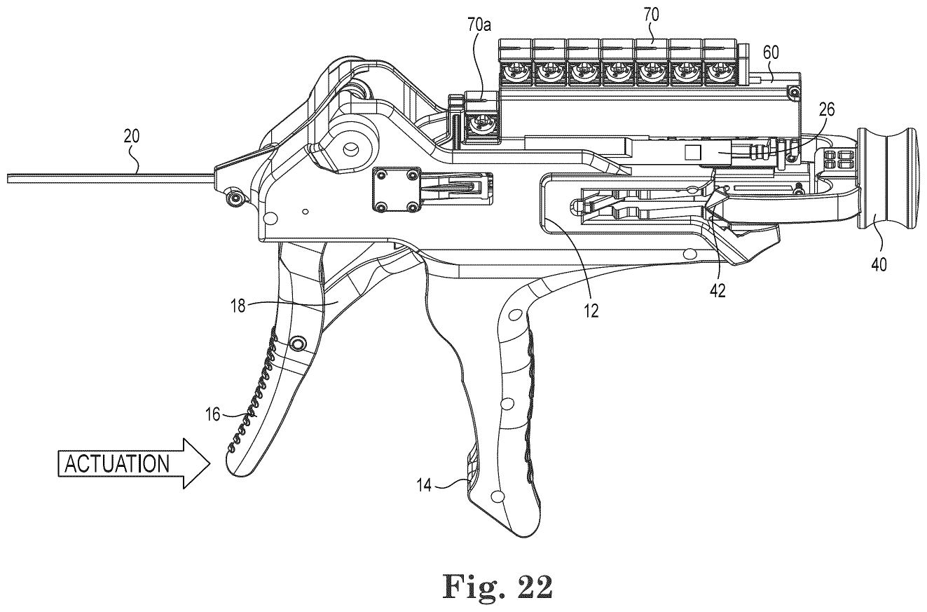

Referring next to FIG. 22, to complete the anchor implantation, the physician will continue toggling the anchor and then pull the working suture tight. Both these steps call for manipulation of the working suture. The bone punch 40 and the inner tube 22 are retracted to avoid interfering with or becoming entangled with the working suture and suture lock, and also to prevent any nicks or cuts in the working suture. To retract the bone punch 40 and inner tube 22, the physician actuates the trigger 16 to again release the bone punch latch and retract the bone punch. Because the cartridge 70a is depressed, when the trigger 16 is actuated and squeezed toward the handle 14, the retracting forces are applied to the upper and lower slides via the cartridge itself. This retracts the bone punch as well as the inner tube from the bone hole. The result is as shown in FIG. 22, with the bone punch, upper slide 26 and magazine 60 pulled back in relation to the housing 12. Due to the trigger spring (FIG. 4B), after the trigger is actuated, the trigger 16 and the lower slide will be pulled in the distal direction, however, the remaining components will stay retracted.

Referring now to FIG. 30, in the bone hole, the working suture 420 is used to pull and toggle the anchor 400 to a desirable orientation within the cancellous bone tissue 512, as shown. During this toggling and subsequent tightening of the sutures and suture lock, the anchor tube 20 is pressed against the tendon 500 to provide support to the bone to avoid breaking through the cortical shell 510, it being noted that the distal end of the anchor tube 20 includes and anchor tube tip 28 so that the working suture 420 is constrained to some extent.

The anchor 400 is worked into its final position as shown in FIG. 31. The anchor 400 is turned about 90 degrees relative to the bone hole 520 and the longitudinal axes of the anchor tube 20. The anchor 400 is then pulled to the inside of the cortical shell 500 by tightening the working suture. Once positioned in this way, with the working suture tight between the anchor 400 and a previous anchor (assuming this is at least the second anchor in an array), the suture lock is tightened, as described relative to FIG. 24. If the anchor 400 is the first in an array, the working suture may be permanently affixed to the anchor prior to implantation, if desired, or, alternatively, once the desired positioning is achieved, the working suture can be locked in place using the suture lock.

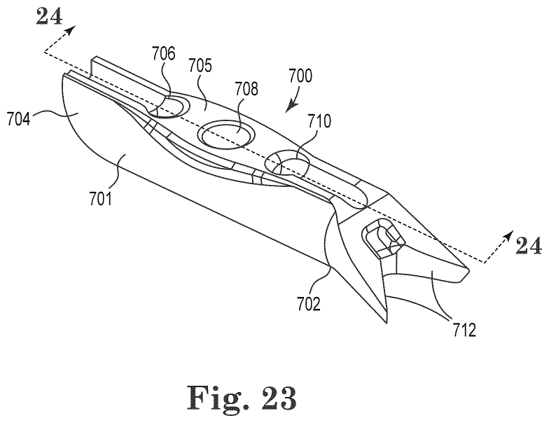

FIGS. 23-25 are a series of illustrations of exemplary toggle bodies or toggle-type anchors that can be used in a procedure for attaching tendon to bone. The illustrations also show a single working suture slidably disposed in passages through the anchor and through a locking loop. The locking loop is configured to have an open position allowing movement of the single working suture, and a closed or locked position that prevents movement of the single working suture.

Referring to FIG. 23, a perspective view of a representative anchor 700 is illustrated. The anchor 700 can be an elongate body 701 having a length defined by a proximal end 702 and a distal end 704. The elongate body 701 can be a generally cylindrical body but other shapes are possible. The anchor 700 has a top surface 705 and a bottom surface (not shown), each of which may have a flat, axially-extending surface that allows room for sutures when the anchor 700 is in a round delivery tube. The length of the anchor 700 is substantially longer than the diameter thereof, allowing the anchor 700 to be inserted lengthwise or axially into a small bone hole. Once inserted, the entire body 701 is pivoted or toggled so that it stays within the bone and has substantially its entire length compressed against material inside the bone. That is, the longitudinal axis of the anchor 700 is rotated or pivoted from the direction used to insert through the bone hole, thereby preventing removal. This approach means that removal would require the anchor itself to fail, rather than simply being released from surrounding tissue, and provides high pullout strength (in some illustrative examples, greater than 600 N before anchor failure when implanted in the array disclosed herein, though the specific force is not intended to be limiting) from an anchor requiring a very small insertion hole (less than about 3 mm). As previously stated and described in detail below, small insertion holes allow much closer placement of anchors in a high-density array.

The anchor 700 can have a length of about 6 mm to about 10 mm in some embodiments. This length gives adequate strength while leaving enough room inside the bone for the high number of anchors implanted. Each anchor is preferably molded or machined from a polymeric material, preferably a high tensile strength material such a poly-ether-ether ketone (PEEK) which is also highly biocompatible. In applications where MRI imaging would not be an issue, metal can be utilized in part or all of the anchor body.

It can be seen that the anchor 700 can include a number of holes or passages through the cross section of the anchor 700. As illustrated, the anchor 700 has a proximal bore or passage 710, a middle passage 708 and a distal passage 706. The use of proximal and distal refers to how the anchor 700 is arranged inside a cartridge as shown above. The passages 706, 708, 710 extend from the top surface 705 to the bottom surface such that the passages 706, 708, 710 extend through the cross section of the elongate body 701. In other embodiments, the anchor may have fewer or more bores or passages, such as having a single bore, two bores, or more than three bores.

The distal end 704 of the anchor 700 has an angled surface. The angled surface creates a longer upper longitudinal surface 705 than lower longitudinal surface. In other words, the upper surface 705 projects a greater distance distally than the lower surface. This is useful during insertion of the anchor 700 because the projecting distal surface plows into cancellous spongy bone when implanted to initiate at least partial rotation of the toggle body during insertion (see FIG. 29). Keeping in mind that the present anchors 700 are preferably implanted through the tendon, it is important that the anchor 700 toggle every time or it may pull out of the bone hole under tension yet not be visible as it will be under the tendon.

The proximal end 702 of the anchor 700 can include one or more projecting fins 712. The illustrated embodiment includes two fins 712. Each fin 712 projects outward and proximally. In some embodiments, as depicted, the fins 712 project downward as they extend proximally. The function of the fins 712 is to aid in toggling and prevent backing-out of the anchor 700. The fins 712 may project outward and/or downward beyond the general maximum cross section or diameter of the elongate body 701. The bone hole in which the implant will be placed is sized to closely match this dimension, as is the inner diameter of a delivery tube used to deliver the anchor 700. During insertion the fins 712 flex inward under compressive force due to contact with the inner diameter of the inner tube, and are then passed into the bone hole.

Once delivered and released from compressive forces of the inner tube, the fins 712 relax to a size greater than the bone hole. In some preferred embodiments, each fin tip extends about an additional 0.5 mm beyond the size of the bone hole through which the anchor is inserted. Such fin tips may also be described as extending about 0.5 mm beyond the maximum outer diameter of the rest of the anchor body, for example, in the range of 0.4 mm to 0.7 mm. This feature provides an added safeguard against the anchor 700 backing out of the bone hole under tension if the anchor 700 has not adequately toggled. Further, the fins 712 are positioned so that tension on the anchor 700 causes the partially toggled anchor to grab cancellous bone and further rotate the anchor.

FIG. 24 show a section view of the anchor 700 along line 24 -24 of FIG. 23, and the working suture and suture lock are added to the drawing. The anchor 700 includes the distal passage 706 and proximal passage 710. The working suture 715 passes from the top surface of the anchor 700 to the bottom surface at the distal passage 706, then along the bottom of the anchor 700 past the middle passage and to the proximal passage 710, then up from the bottom surface to the top surface of the anchor 700.

The suture lock 716 is shown with a locking loop 118, secured by a sliding knot 720, a break knot 722, and a free end 721. The free end 721 extends to the spool in the cartridge, as shown in FIG. 14 (suture lock 410 extends to spool 306). The suture lock 716 passes through the middle passage of the anchor 700. The middle passage optionally defines a shelf at 714, such that the middle passage includes a larger diameter upper portion and a smaller diameter lower portion, allowing the slip knot 720 to be positioned in the upper portion of the middle passage, above shelf 714, and preventing the suture lock 716 from passing entirely through the anchor 700. The locking loop 718 encircles a portion of the working suture 715.

As shown, the working suture 715 can slide through each of the proximal passage 710 and distal passage 706, allowing the anchor 700 to floss along the working suture 715 during the implantation procedure. Once the anchor 700 is positioned in a bone at a desired location and orientation, the physician can then lock the anchor and working suture 715 by pulling on the free end of the suture lock 716. This force on the free end 721 causes the locking loop 718 to collapse onto the working suture 715. The sliding knot 720 prevents release of the collapsed locking loop 718. Further, collapsing the locking loop 118 compresses the cross section of the working suture 715. A particularly preferred knot at 720 is a 4-throw uni knot. However, other slidable knots 720 may be used, as desired. If desired, the underside of the anchor 700 may include an indentation or channel into which the working suture 715 may be pulled by the locking loop 718.

The working suture 715 is preferably a braided multistrand suture having a compressible cross-sectional area that reduces by at least about 25% when the locking loop 718 is tightened during use. The working suture 715 can be a round and/or braided No. 2 suture in some embodiments. Other size and type sutures may be used.

After the sliding knot 720 is tightened, continued pulling force applied to the free end 721 of the suture lock will cause a preferential point of failure in the suture lock 716 (such as the break knot 722 or nick, which may take the place of break knot 722) to break. This leaves only a short portion of the suture lock 716 attached to the anchor 700. After breaking at the break knot 722, the rest of the proximal portion of the suture lock 716 can be discarded. In some examples, a more proximal portion of the suture lock is secured to a cartridge, so that a physician may cause the suture lock to break as shown by pulling on the cartridge itself. In an example, the preferential point of failure is designed to allow tightening of the locking loop 718 onto the working suture 715 before the failure occurs. For example, the locking loop and the preferential point of failure may be configured for breaking under a pull strength in the range of 3-10 pounds of force, more preferably, 5-7 pounds of force, or more or less as desired. The pull strength needed to tighten the locking loop 718 onto the working suture may be less than the pull strength needed for breaking the preferential point of failure in some examples by, for example, an amount in the range of 0.5 to 3 pounds, or 0.75 to 2 pounds, or about 1 pound.

The working suture and suture lock 715, 718 may be, for example, made of natural material such as silk and/or synthetic materials such as polyglycolic acid, polylactic acid, and polydioxanone, each of which are known for use as absorbable sutures, and/or nylon and polypropylene, which are typically non-absorbable. Various coatings, including antimicrobial, anti-wicking or lubricious coatings may be applied as well. More broadly, the working suture and suture lock 715, 718 may include any item that can be used to couple together objects in a surgical environment, such as any sufficiently biocompatible metal, natural material, plastic or other artificial material adapted for use in a surgical procedure. Monofilaments or more complex structures including braids, weaves, windings, twisted threads, coated or multilayer member, etc. may be used.

In FIG. 25 a pre-strung array 801 of individual anchors 800 is depicted. The anchors 800 may be similar in form and function to the anchor 700 of FIG. 23-24. The array has four anchors 800 as a representative chain. It is believed chains of 4 to 12 anchors would be useful in tendon repair procedures such as rotator cuff repair. One particular embodiment includes 8 anchors in an array.

A working suture 820 is pre-threaded through the series of anchors 800 to assure that they will toggle as desired and tension to form the stitch when the suture is tightened. The illustration shows the first anchor 802 to be implanted followed by the second anchor 804, then the third anchor 806 and finally the fourth anchor 808. With this order of implantation understood, the working suture 820 has been pre-threaded down through the top of the proximal hole 810 and back up through the distal hole 811 of the first anchor 802. The working suture 820 then continues to the second anchor 804 where it is threaded down through the proximal hole 812 and back up through the distal hole 813 of the second anchor 804. The working suture 820 then continues to the third anchor 806 where it enters the top of the proximal hole 814 and back up the distal hole 815 of the third anchor 806. The working suture then continues to the fourth anchor 808 where it enters the top of the proximal hole 816 and passes up through the bottom of distal hole 817 of the fourth anchor 808. If the array were more than four anchors, the pre-threading would continue as described for each subsequent anchor.

To implant the array, the first anchor 802 would be positioned at a first location, using the methods illustrated with FIGS. 17-22 and FIGS. 26-31. The working suture 820 would then be locked into position at the first anchor 802 using a suture lock as described in relation to FIG. 24. Alternatively, the working suture 820 may be permanently attached at the first anchor 802.

Next, the second anchor 804 would be positioned at a second location, using the methods illustrated with FIGS. 17-22 and FIGS. 26-31. The portion of the working suture 820 between the first anchor 802 and second anchor 804 would then be tightened by pulling on the portion of the working suture 820 between the second anchor 804 and the third anchor 806 (the third anchor would still be in a cartridge in a magazine on the implantation tool). Once the portion of the working suture 820 between the first anchor 802 and second anchor 804 is tightened to the satisfaction of the physician, the suture lock at the second anchor 804 would be activated/engaged as described in relation to FIG. 24. Once the suture lock is applied at the second anchor 804, a secure, tight stitch between the first and second anchors has been created. The process of implanting an anchor, tightening the working suture, and engaging a suture lock would be repeated for each subsequent anchor in the array.

A result of this implantation sequence is illustrated in FIG. 32. A bone 900 has a tendon 902 secured thereon with one or more arrays of suture anchors, as shown at 904. The arrays may be implanted in a single line, or may zig-zag, as shown, as needed for the particular bone and tendon, in the judgement of the physician. In an illustrative example, the bone 900 is the humeral head, and the tendon 902 is the supraspinatus tendon. Other bones and tendons may be used instead.

Various alternatives and further details for anchors, anchor arrays, implantation tools, and methods associated with each can be found in U.S. Pat. Nos. 11,382,611, 11,375,991, 11,389,154, 11,382,612, 11,389,290, 11,375,992, 11,375,995, 11,382,613, and 12,226,307, as well as US Pre-Grant Publications 20220323198, 20220378409, and 20220323200, the disclosures of which are incorporated herein by reference.

The anchor delivery tool 10 may be used with other anchors. For example, the tool can be described as facilitating a two-step use of the bone punch, with a first step having the bone punch passed down the anchor tube and inner tube to the distal tips thereof, and then tapped or pounded to make a bone hole. The trigger can be used to release the bone punch from the bone hole and to release the bone punch latch from the housing. The bone punch is then retracted to allow the cartridge to be depressed into position, linking the upper and lower slides together and aligning an anchor with the lumens in the upper slide and inner tube. The bone punch can then be used to advance the anchor into the bone hole, passing through the cartridge. The trigger is then used to retract the bone punch and inner tube by forces applied to the upper and lower slides. Each of these steps can be performed using hard anchors or soft anchors, as desired. Indeed, it is envisioned that a range of different anchor types may be implanted using the tool as shown and described herein.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. An implantation tool for placement of a bone anchor array comprising:
a housing with a handle and a trigger;
an anchor tube having an anchor tube proximal end and an anchor tube distal end, the anchor tube proximal end attached to the housing;
an upper slide moveably positioned in the housing, the upper slide including a cartridge opening having a ledge therein, the upper slide moveable between a delivery position and a retracted position;
a magazine holding a plurality of cartridges including at least a first cartridge and a second cartridge, the first cartridge being distal of the second cartridge in the magazine, each cartridge having a shoulder at a lower portion thereof and an anchor space open at proximal and distal ends, the anchor space containing an anchor of the bone anchor array, the upper slide configured to receive the magazine such that the distalmost cartridge is positioned over the cartridge opening, wherein two positions are defined for the distalmost cartridge:
a first position in which the shoulder is positioned above the ledge; and
a second position in which the distalmost cartridge is depressed into the cartridge opening with the shoulder abutting the ledge.

2. The implantation tool of claim 1, wherein the magazine includes a front stop, a plunger and a spring, the spring urging the plunger distally to press the cartridges toward the front stop.

3. The implantation tool of claim 2, wherein the front stop comprises a cartridge retainer and each cartridge includes a cartridge indent, the cartridge retainer positioned to engage the cartridge indent of a distalmost cartridge in the magazine to hold the distalmost cartridge in the magazine in the first position until the distalmost cartridge in the magazine is manually depressed into the second position.

4. The implantation tool of claim 2, wherein each cartridge has a distal side with a cartridge indent, and a proximal side with a cartridge projection, positioned such that the cartridge projection of the first cartridge is engaged with the cartridge indent of the second cartridge to hold the first cartridge in the first position until manually depressed into the second position.

5. The implantation tool of claim 2, wherein each cartridge has a distal side with a cartridge projection, and a proximal side with a cartridge indent, positioned such that the cartridge indent of the first cartridge is engaged with the cartridge projection of the second cartridge to hold the first cartridge in the first position until manually depressed into the second position.

6. The implantation tool of claim 2, wherein each cartridge in the magazine is removeable from the magazine when in the second position, and the spring and plunger are adapted to apply pressure to hold a distalmost remaining cartridge against the front stop as each successive cartridge is removed from the magazine.

7. The implantation tool of claim 1, wherein the anchor space is generally cylindrical having a top slot.

8. The implantation tool of claim 1, wherein when the first cartridge is in the second position, the first cartridge blocks the distal end of the anchor space of the second cartridge.

9. The implantation tool of claim 1, wherein the housing has a distal end at which the anchor tube is attached, and a proximal end opposed to the distal end, and width, and the magazine has a width, and the width of the magazine is less than the width of the housing.

10. The implantation tool of claim 1, wherein the magazine is centrally located within the width of the housing.

11. A method of placing an anchor in a bone using an implantation tool, the method comprising:
using an implantation tool having a housing with a proximal end and a distal end, with an anchor tube extending from the distal end of the housing to an anchor tube distal tip, an upper slide moveable relative to the housing and adapted to receive a magazine containing cartridges, the upper slide having a cartridge opening and a first lumen extending proximally from the cartridge opening and a second lumen extending distally from the cartridge opening, and a bone punch having a bone punch distal tip, a shaft, a bone punch latch, and a bone punch handle, placing a magazine in the upper slide to position a cartridge held by the magazine over the cartridge opening;
positioning the distal tip of the anchor tube at a desired location for placing the anchor;
advancing the bone punch into the anchor tube until the bone punch distal tip exits the bone punch and forms a bone hole;
retracting the bone punch from the bone hole a first time, through the anchor tube and into the housing until the bone punch distal tip is in a lumen of the upper slide proximal of the cartridge opening;
depressing the cartridge into the cartridge opening;
advancing the bone punch through the cartridge to push the anchor from the cartridge through the anchor tube and into the bone hole; and
retracting the bone punch from the bone hole a second time.

12. The method of claim 11, wherein the cartridge holds the anchor in an anchor space having a proximal open end and a distal open end, and the step of depressing the cartridge into the cartridge opening positions the proximal open end in alignment with the first lumen of the upper slide, and the open distal end in alignment with the second lumen of the upper slide.

13. The method of claim 11, wherein the upper slide includes an inner tube attached thereto, the inner tube extending distally from the upper slide into the anchor tube and having an inner tube lumen aligned with the second lumen of the upper slide, such that the step of advancing the bone punch through the cartridge to push the anchor from the cartridge through the anchor tube and into the bone hole includes pushing the anchor through the inner tube lumen.

14. The method of claim 13, wherein the upper slide is moveable relative to the housing, such the inner tube is moveable relative to the anchor tube, wherein:
advancing the bone punch into the anchor tube until the bone punch distal tip exits the bone punch and forms a bone hole includes advancing the upper slide relative to the housing and advancing the inner tube relative to the anchor tube such that the inner tube extends into the bone hole;
retracting the bone punch from the bone hole a first time is performed without removing the inner tube from the bone hole; and
retracting the bone punch from the bone hole a second time includes removing the inner tube from the bone hole.

15. The method of claim 11, wherein the anchor has at least one bore through which a working suture passes and at least one bore through which a suture lock extends, the suture lock having a loop extending around the working suture and a free end secured to the cartridge, the cartridge containing a spool on which the free end is wound.

16. A combination cartridge, bone anchor for implantation in a bone, and suture lock, the suture lock having a free end and a locking loop, the cartridge configured for holding the bone anchor for implantation in a bone, the cartridge comprising a body having a distal side, a proximal side, and an anchor space extending from an open end at the distal side to an open end at the proximal side, the anchor space having a slot extending upward therefrom toward an upper end of the body, the cartridge holding therein a spool for receiving the free end of a suture lock, the bone anchor having a bore therethrough having a first end and a second end, wherein the suture lock is positioned in the bore with the locking loop extending out of the first end of the bore and the free end extending up to the gap and then to the spool out of the second end of the bore.

17. The combination of claim 16, wherein the cartridge has a cartridge gap along one side of the slot, and a protrusion extending laterally from the cartridge adjacent the cartridge gap, the protrusion for securing a position of the cartridge relative to an implantation tool for use therewith.

18. The combination of claim 16, wherein the cartridge is configured to be held in a magazine, the magazine having an upper slot, the cartridge having a neck sized to fit in the upper slot, the spool being positioned above the neck.

19. The combination of claim 18, wherein the cartridge includes an indent configured to interact with either another cartridge or a front stop of a magazine to hold the cartridge in a first position therein until manually depressed to a second position.

20. The combination of claim 16, wherein the cartridge has a shoulder at a lower portion thereof, the shoulder configured to limit downward movement of the cartridge into a housing of an implantation tool configured for use with the cartridge.
